(19) United States
(12) Patent Application Publication
(10) Pub. No.: US 2011/0136113 A1
(43) Pub. Date: Jun. 9, 2011

(54) MARKER FOR DETECTING IL-17-PRODUCING HELPER T CELL, AND METHOD FOR DETECTING IL-17-PRODUCING HELPER T CELL

(75) Inventors: Hitoshi Uga, Kobe (JP); Masakazu Kadowaki, Kobe (JP); Yoshiaki Miyamoto, Kobe (JP); Masafumi Ikeda, Akashi (JP); Satoshi Tanaka, Kobe (JP); Masatoshi Yanagida, Kobe (JP); Takahiro Okazawa, Kobe (JP); Hirokazu Kurata, Kobe (JP)

(73) Assignee: SYSMEX CORPORATION, Kobe, Hyogo (JP)

(21) Appl. No.: 13/058,755

(22) PCT Filed: Aug. 26, 2009
(86) PCT No.: PCT/JP2009/064856

§ 371 Date: Feb. 11, 2011

(30) Foreign Application Priority Data
Aug. 27, 2008 (JP) 2008-218196

(51) Int. Cl. C12Q 1/68; C07H 21/04; C07K 14/435; C12Q 1/02

(52) U.S. Cl. 435/6.1; 536/23.5; 530/350; 435/29

(57) ABSTRACT

Disclosed is at least one polynucleotide marker or protein marker which enables the specific detection of an IL-17-producing helper T cell (a Th17 cells). Also disclosed is a method for detecting a Th17 cell, which is characterized by comprising detecting the occurrence of the above-mentioned at least one marker.

### TECHNICAL FIELD

The present invention relates to a marker for detecting IL-17-producing helper T-cells (hereinafter referred to as “Th17 cells”) and a method for detecting Th17 cells.

### BACKGROUND ART

Rheumatoid arthritis (hereinafter referred to as “RA”) is the systemic inflammatory autoimmune disease whose main clinical symptom is arthritis. The state of RA is diagnosed by visual procedures or rational symptoms such as joint pain or observations on the extent of swelling or bone X-ray. However, no quantitative index has been established. Thus, no quantitative method for continuously monitoring the treatment effects of RA has been established under the current state of the art.

The detailed pathogenesis of RA has not been elucidated. It is considered that bacterial infections and the like trigger an inflammation in joint tissues via complicated networks of immunocytes and cytokines.

Helper T-cells are mainly responsible for immune reactions. Immature helper T-cells or naïve T-cells are differentiated into helper T-cells when an antigen is presented by antigen-presenting cells. When specific cytokines are present at this time, naïve T-cells are differentiated into four types of the cells, which are helper T-cells producing interferon (IFN)-γ (Th1 cells), helper T-cells producing interleukin (IL)-4 (Th2 cells), helper T-cells producing IL-17 cells (Th17 cells) and regulatory T-cells having immunosuppressive effects (Treg cells).

It has been shown that among these helper T-cells, Th17 cells can be involved in the onset of RA.

For example, Japanese Unexamined Patent Publication No. 2000-186046 (Patent Literature 1) suggests that IL-17 is deeply involved in the formation of pathological condition and in particular in joint and bone deformities because the level of IL-17 which is produced by Th17 cells is significantly higher in synovial fluid of RA patients than in that of the patients of osteoarthritis and T-cells in synovial tissue from RA patients include IL-17 positive cells. Patent Literature 1 also discloses that IL-17 can be used as a diagnostic marker of RA.

Further, Japanese Unexamined Patent Publication No. 2007-506100 (Patent Literature 2) discloses that the analysis of cytokines in peripheral blood serum of RA patients revealed that the levels of IFN-γ, IL-1β, TNF-α, G-CSF, GM-CSF, IL-6, IL-4, IL-10, IL-13, IL-5 and IL-7 were significantly high and the levels of IL-2, CXCL8/IL-8, IL-12 and CCL2/MCP-1 were not high in RA patients.

According to the studies by Ivanov et al. (Cell, 2006, 126, p. 1121-1133; Non Patent Literature 1), Stumhofer et al. (Nature Immunology, 2006, vol. 7, p. 937-945; Non Patent Literature 2), and Wilson et al. (Nature Immunology, 2007, vol. 8, p. 950-957; Non Patent Literature 3), the following facts have been shown about Th17 cells:
a nuclear receptor called RORγt has an important role in the differentiation of Th17 cells;
IL-6, IL-23 and TGF-β induce the differentiation of naïve T-cells to Th17 cells;
Th17 cells express IL-17A, IL-17F, IL-6, IL-22, IL-26, TNF, IFN-γ and CCL20; and
IL-23 receptor and IL-12 receptor f3 are located on the surface of Th17 cells.

In the above Non Patent Literatures 1 to 3, the amount of IL-17 is measured by enzyme linked immunosorbent assay (ELISA) using antibodies specific to IL-17.

The relations between Th17 cells and immunological diseases such as RA may be more deeply understood by establishing a method which is able to not only measure the amount of IL-17 but also detect Th17 cells per se.

### CITATION LIST

### Patent Literatures

Patent Literature 1: Japanese Unexamined Patent Publication No. 2000-186046

Patent Literature 2: Japanese Unexamined Patent Publication No. 2007-506100

### Non Patent Literatures

Non Patent Literature 1: Ivanov et al., “The Orphan Nuclear Receptor RORγt Directs the Differentiation Program of Proinflammatory IL-17+ T Helper Cells”, Cell, 2006, 126, p. 1121-1133

Non Patent Literature 2: Stumhofer et al., “Interleukin 27 negatively regulates the development of interleukin 17-producing T helper cells during chronic inflammation of the central nervous system” Nature Immunology, 2006, vol. 7, p. 937-945

Non Patent Literature 3: Wilson et al., “Development, cytokine profile and function of human interleukin 17-producing helper T cells” Nature Immunology, 2007, vol. 8, p. 950-957

### SUMMARY OF INVENTION

### Technical Problem

The inventors aimed to identify molecular markers that make it possible to specifically detect Th17 cells.

### Solution to Problem

First, the inventors identified genes which are specifically expressed in Th17 cells differentiated from naïve T cells isolated from the spleen of mice. The inventors then identified, among thus identified genes, the genes which are highly expressed in model mice of three autoimmune diseases in which Th17 cells are considered to be involved, i.e. arthritis, inflammatory bowel disease and encephalomyelitis, and completed the present invention.

Thus, the present invention provides a polynucleotide marker for detecting IL-17-producing helper T cells (Th17 cells), which is a polynucleotide derived from at least one gene selected from:

a gene encoding a protein present in cells (intracellular protein) represented by Id2 (inhibitor of DNA binding 2), Msc (musculin), Nfat5 (nuclear factor of activated T-cells 5), Nfkbiz (nuclear factor of kappa light polypeptide gene enhancer in B-cells inhibitor, zeta), Plekho1 (pleckstrin homology domain containing, family O member 1), Runx2 (runt related transcription factor 2), Tcf12 (Transcription factor 12), Vax2 (ventral anterior homeobox containing gene 2), Zc3h12a (zinc finger CCCH type containing 12A), Tnik (TRAF2 and NCK interacting kinase), B3gnt8 UDP-G1cNAc:betaGal (beta-1,3-N-acetylglucosaminyltransferase 8), Gcnt2 (glucosaminyl (N-acetyl) transferase 2, I-branching enzyme), 1190003J15Rik (RIKEN cDNA 1190003J15 gene), Cybb (cytochrome b-245, beta polypeptide), Rab32 (RAB32, member RAS oncogene family), Bcl2a1 (B-cell leukemia/lymphoma 2 related protein A1), Bcl7a (B-cell CLL/lymphoma 7A), Cmah (cytidine monophospho-N-acetylneuraminic acid hydroxylase), Dab2 (disabled homolog 2), Fcer1a (Fc receptor, IgE, high affinity I, alpha polypeptide), Nlrp4c (NLR family, pyrin domain containing 4C), Rbp1 (retinol binding protein 1, cellular), Stab1 (stabilin 1), Tubb2c (tubulin, beta 2c), Upp1 (uridine phosphorylase 1), Rc3h2 (ring finger and CCCH-type zinc finger domains 2), Ddx6 (DEAD (Asp-Glu-Ala-Asp) box polypeptide 6), Actr1a (ARP1 actin-related protein 1 homolog A) or Cyp1b1 (cytochrome P450, family 1, subfamily b, polypeptide 1);

a gene encoding a cell membrane protein represented by March2 (membrane-associated ring finger (C3HC4) 2), Abcal (ATP-binding cassette, sub-family A (ABC1), member 1), Ccr2 (chemokine (C-C motif) receptor 2), Cd160 (CD160 antigen), Cd1d2 (CD1d2 antigen), Cd300a (CD300A antigen), Clec4n (C-type lectin domain family 4, member n), Cnr2 (cannabinoid receptor 2 (macrophage)), Cr1l (complement component (3b/4b) receptor 1-like), Crtam (cytotoxic and regulatory T cell molecule), Csf2rb (colony stimulating factor 2 receptor, beta, low-affinity (granulocyte-macrophage)), Cxcr6 (chemokine (C-X-C motif) receptor 6), Cd1631l (CD163 molecule-like 1), Fcgr2b (Fc receptor, IgG, low affinity IIb), H2-Ea (histocompatibility 2, class II antigen E alpha), Il27ra (interleukin 27 receptor, alpha), Itgae (integrin, alpha E, epithelial-associated), Klrb1f (killer cell lectin-like receptor subfamily B member 1F), Klrc1 (killer cell lectin-like receptor subfamily C, member 1), Klrc2 (killer cell lectin-like receptor subfamily C, member 2), Klrd1 (killer cell lectin-like receptor, subfamily D, member 1), Pdpn (podoplanin), Slc2a6 (solute carrier family 2 (facilitated glucose transporter), member 6), Tmem176a (transmembrane protein 176A), Tnfrsf14 (tumor necrosis factor receptor superfamily, member 14 (herpesvirus entry mediator)), Tnfrsf25 (tumor necrosis factor receptor superfamily, member 25), Umodl1 (uromodulin-like 1), Vcam1 (Vascular cell adhesion molecule 1), Ebi2 (Epstein-Barr virus induced gene 2), Tmem176b (transmembrane protein 176B) or I17r (interleukin 7 receptor alpha chain);

a gene encoding a secretory protein represented by Acpp (acid phosphatase, prostate), Bmp1 (bone morphogenetic protein 1), Bpil2 (bactericidal/permeability-increasing protein-like 2), Ccl3 (chemokine (C-C motif) ligand 3), Crispld2 (cysteine-rich secretory protein LCCL domain containing 2), Ctsc (cathepsin C), Gzmc (granzyme C), Il2 (interleukin 2), Lum (lumican), Ly86 (lymphocyte antigen 86), Lyz1 (lysozyme 1), Mcpt1 (mast cell protease 1), Mmp10 (matrix metallopeptidase 10), Mmp13 (matrix metallopeptidase 13), Prg4 (proteoglycan 4 (megakaryocyte stimulating factor, articular superficial zone protein)), Rln1 (Relaxin 1), S100a8 (S 100 calcium binding protein A8 (calgranulin A)), Tgfbi (transforming growth factor, beta induced), Timp1 (tissue inhibitor of metalloproteinase 1) or Apol7b or Apol7e (apolipoprotein L 7b or apolipoprotein L 7e); and

a gene represented by 1200015M12Rik (RIKEN cDNA 1200015M12 gene), 1200016E24Rik (RIKEN cDNA 1200016E24 gene), 1300007F04Rik (RIKEN cDNA 1300007F04 gene), 2010002N04Rik (RIKEN cDNA 2010002N04 gene), 2900073C17Rik (RIKEN cDNA 2900073C17 gene), 4930480G23Rik (RIKEN cDNA 4930480G23 gene), 4933424M12Rik (RIKEN cDNA 4933424M12 gene), 5330403D14Rik (RIKEN cDNA 5330403D14 gene), 5430434G16Rik (RIKEN cDNA 5430434G16 gene), 5830444B04Rik (RIKEN cDNA 5830444B04 gene), 9630026M06Rik (RIKEN cDNA 9630026M06 gene), A630023P12Rik (RIKEN cDNA A630023P12 gene), AI426953 (expressed sequence AI426953), AU015263 (expressed sequence AU015263), C030044O21Rik (RIKEN cDNA C030044O21 gene), C230085N15Rik (RIKEN cDNA C230085N15 gene), Car13 (carbonic anhydrase 13), Hmga2-ps1 (high mobility group AT-hook 2, pseudogene 1), LO0552902 (hypothetical LO0552902), Rbm10 (RNA binding motif protein 10), Rrad (Ras-related associated with diabetes), Sbno2 (strawberry notch homolog 2), Speer1-ps1 (spermatogenesis associated glutamate (E)-rich protein 1, pseudogene 1), AK086919 (expressed sequence AK086919), BG071091 (expressed sequence BG071091), AK037075 (expressed sequence AK037075), AK048317 (expressed sequence AK048317), AK081530 (expressed sequence AK081530), AK143436 (expressed sequence AK143436), AW538350 (expressed sequence AW538350), AK138895 (expressed sequence AK138895), AI593283 (expressed sequence AI593283), BM219171 (expressed sequence BM219171), BB204715 (expressed sequence BB204715), AI447357 (expressed sequence AI447357), AK047185 (expressed sequence AK047185), AW491352 (expressed sequence AW491352), AK037546 (expressed sequence AK037546), BG072223 (expressed sequence BG072223), BB283635 (expressed sequence BB283635), BB800733 (expressed sequence BB800733), AK136422 (expressed sequence AK136422), BB309694 (expressed sequence BB309694), AI661001 (expressed sequence AI661001), AK080134 (expressed sequence AK080134), AA982044 (expressed sequence AA982044), AI447438 (expressed sequence AI447438), BB204715 (expressed sequence BB204715), AK041551 (expressed sequence AK041551), AI448729 (expressed sequence AI448729), BE951087 (expressed sequence BE951087), AK157595 (expressed sequence AK157595), AK085158 (expressed sequence AK085158), AK028862 (expressed sequence AK028862), BG076280 (expressed sequence BG076280), BM115076 (expressed sequence BM115076), AK037590 (expressed sequence AK037590), GENSCAN00000037577 (expressed sequence GENSCAN00000037577), AK037171 (expressed sequence AK037171), AK141370 (expressed sequence AK141370), AK031033 (expressed sequence AK031033), AW120969 (expressed sequence AW120969), AI605450 (expressed sequence AI605450), AK138320 (expressed sequence AK138320), AK149443 (expressed sequence AK149443), AK090273 (expressed sequence AK090273), BB043576 (expressed sequence BB043576) or AK036007 (expressed sequence AK036007).

The present invention also provides a protein marker for detecting Th17 cells consisting of at least one protein encoded by the above gene.

The present invention further provides a method for detecting Th17 cells comprising detecting the presence of at least one polynucleotide marker for detecting Th17 cells or at least one protein marker for detecting Th17 cells as described above in a sample containing cells.

Th17 cells can be sufficiently detected by detecting at least one marker of the present invention. It is considered that Th17 cells can be detected with higher reliability by detecting two or more markers.

### Advantageous Effect of Invention

Th17 cells can be specifically detected by detecting the present polynucleotide or protein marker. Accordingly, Th17 cells can be isolated by using the present marker. For example, Th17 cells can be specifically detected in samples containing cells such as tissues obtained from patients by using the present marker. Therefore, the potential morbidity of the patients to the autoimmune diseases may be detected in which Th17 cells may be involved such as RA, inflammatory bowel disease and multiple sclerosis.

The expression level of the polynucleotide marker or protein marker of the present invention may vary in each stage of autoimmune diseases, i.e. during early stage, climax and convalescence. Thus, the pathological condition may be monitored by measuring the expression level of the present marker by means of ELISA, flow cytometry (FCM), microarray and the like.

### BRIEF DESCRIPTION OF DRAWINGS

FIG. 1 shows the expression level of Il7r gene in Th1, Th2, Treg and Th17 cells derived from C57/BL6 mice.

FIG. 2 shows TIMP-1 protein concentration in the culture supernatants of Th1, Th2, Treg and Th17 cells which have been differentiation cultured from naïve T-cells derived from BALB/c mice.

FIG. 3 shows two-dimensional scattergrams obtained from the analysis of Th17 cells on flow cytometer using anti-CD4, anti-TNFRSF14, anti-IL7R, anti-PDPN and anti-CCR6 antibodies.

### DESCRIPTION OF EMBODIMENTS

The present polynucleotide marker for detecting Th17 cells is a polynucleotide derived from the above gene, namely is the gene per se (DNA), mRNA, cDNA or cRNA. The above polynucleotide marker is selected from the polynucleotide derived from the gene and variants and fragments thereof.

The polynucleotide marker is the polynucleotide, variant or fragment thereof which has been found to be specifically present in Th17 cells rather than in Th1, Th2 or Treg cells. The polynucleotide marker has also been found to be specifically expressed in model mice of the diseases in which Th17 cells are involved. Therefore, by detecting at least one polynucleotide marker as above, Th17 cells can be distinguished from Th1, Th2 and Treg cells and specifically identified.

The present invention also encompasses the use of the polynucleotide derived from the above gene and the protein encoded by the above gene as a marker for detecting Th17 cells.

As used herein, the term “gene” has the same meaning as that is commonly recognized in the art, and refers to a part of a genome which is transcribed into mRNA and translated into a protein.

As used herein, the expression that a polynucleotide is “specifically expressed” in Th17 cells means that the expression level of the polynucleotide in Th17 cells is significantly higher than the expression level of the polynucleotide in cells other than Th17 cells. Specifically, it means that the expression level of the polynucleotide in Th17 cells is about 1.5 times or more, more preferably three times or more higher than the expression level of the polynucleotide in cells other than Th17 cells. More preferably, the expression level of the polynucleotide in Th17 cells is about 1.5 times or more, more preferably about three times or more higher than the expression level of the polynucleotide in Th1, Th2 and Treg cells.

As used herein, the expression that a polynucleotide is “specifically expressed” in a disease model mouse means that the expression level of the polynucleotide in the disease model mouse tissue is significantly higher than the expression level of the polynucleotide in a healthy mouse tissue. Specifically, it means that the expression level of the polynucleotide in the disease model mouse tissue is about two times or more, more preferably three times or more higher than the expression level of the polynucleotide in the healthy mouse tissue.

The nucleotide sequences of the present polynucleotide markers are already known. They can be obtained from, for example, Unigene or Nucleotide (databases provided by National Center for Biotechnology Information (NCBI) of National Library of Medicine). The information on nucleic acid sequences of the present polynucleotide markers may be obtained from the above databases with using the code numbers shown under “Annotation Mapped Transcripts” in Table 4 below, for example.

As used herein, “variant” of a polynucleotide means a polynucleotide into which a mutation has been introduced that does not alter the nature of the protein encoded by the above gene. Such mutation includes a deletion, substitution and addition of one or more nucleotides in the known nucleic acid sequence of the above gene.

The variant has generally at least 80%, more preferably at least 85%, further preferably at least about 90% and particularly preferably at least 95% homology with the known nucleic acid sequence of the above gene.

As used herein, the “homology” of nucleic acid and amino acid sequences means the value calculated in BLASTN, BLASTP, BLASTX or TBLASTN (e.g. available from http://www.ncbi.nlm.nih.gov) with default settings.

Th17 cells can also be detected by detecting the protein encoded by the gene which is the polynucleotide marker of the present invention. Thus, the present invention also provides the protein marker for detecting Th17 cells consisting of at least one protein encoded by the above gene.

The information on amino acid sequences of such protein markers can be obtained based on the nucleic acid sequences of the polynucleotide markers obtained from Unigene and the like. It can also be obtained from databases provided by NCBI and the like.

The protein marker for detecting Th17 cells may be selected from at least one protein encoded by the above genes, functionally equivalent variants thereof and fragments thereof.

“Functionally equivalent variant” of a protein means a protein into which a mutation has been introduced that does not alter functions of the protein. Such mutation includes a deletion, substitution and addition of one or more amino acids in the known amino acid sequence of the above protein.

The functionally equivalent variant of the protein has generally at least 80%, more preferably at least 85%, further preferably at least about 90% and particularly preferably at least 95% homology with the known amino acid sequence of the protein.

A molecule that can specifically hybridize to the polynucleotide marker can be used for the detection of the marker, making it useful as a probe for detecting Th17 cells. The probe may be a nucleic acid probe such as DNA or RNA, or a peptide probe that can specifically hybridize to the polynucleotide marker. The probe for detecting Th17 cells is preferably a nucleic acid probe, particularly a DNA probe for detecting the polynucleotide marker.

As used herein, “a molecule that can specifically hybridize to” the polynucleotide marker means a molecule that can form a duplex with the polynucleotide marker under a stringent condition.

As used herein, “stringent condition” means the condition under which the probe for detecting Th17 cells can hybridize to the target polynucleotide marker with a detectably higher extent than it does to a polynucleotide other than the target polynucleotide marker (e.g. in an extent that is at least more than two times of the background). The stringent condition generally depends on the sequences and varies depending on the various conditions. Generally, the stringent condition is selected so that it is about 5° C. lower than a thermal melting point (Tm) of the specific sequence under a certain ionic strength and pH. This Tm is the temperature at which 50% of the complementary probe hybridizes to the target polynucleotide sequence in equilibrium under a certain ionic strength, pH and nucleic acid composition.

Such condition may be the one which is used in common hybridization techniques between polynucleotides such as PCR, microarray or Southern blotting. Specifically, it may be a condition of pH 7.0 to 9.0, a salt concentration of lower than about 1.5M Na-ion, more specifically about 0.01 to 1.0 M Na-ion concentration (or other salt) and a temperature of at least about 30° C. More specifically, the stringent condition in microarray techniques includes the hybridization at 37° C. in 50% formamide, 1M NaCl and 1% SDS and washing at 60 to 65° C. in 0.1×SSC. The stringent condition in PCR techniques includes a condition of pH 7 to 9, 0.01 to 0.1 M Tris-HCl, 0.05 to 0.15 M potassium ion concentration (or other salt) and at least about 55° C.

The sequence of the nucleic acid probe for detecting Th17 cells can be appropriately selected by a person skilled in the art based on the common technical knowledge in the art and the sequence of the polynucleotide marker so that it can specifically hybridize to the polynucleotide marker.

The nucleic acid probe for detecting Th17 cells can be designed by using, for example, a commonly available primer designing software (e.g. Primer3 (available from http://frodo.wi.mit.edu/cgi-bin/primer3/primer3.cgi) or DNASIS Pro (Hitachi Software Engineering Co., Ltd.)).

The nucleic acid probe for detecting Th17 cells can be prepared according to the polynucleotide synthesis methods which are well-known in the art.

The nucleic acid probe for detecting Th17 cells may be labeled with a labeling substance normally used in the art. The labeled nucleic acid probe for detecting Th17 cells allows an easy detection of the polynucleotide marker for detecting Th17 cells, namely of Th17 cells.

The labeling substance may be the one generally used in the art including radioisotopes such as ³²P, fluorescent substances such as fluorescein, enzymes such as alkaline phosphatase and horseradish peroxidase, and biotin.

Th17 cells can be specifically detected by using one nucleic acid probe for detecting Th17 cells or two or more of the nucleic acid probes in combination.

The nucleic acid probe for detecting Th17 cells may include a set of two or more primers for amplifying the polynucleotide marker by PCR technique, for example.

The present invention also provides a microarray for detecting Th17 cells which comprises a solid phase capable of binding to a nucleic acid, onto which the above nucleic acid probe is immobilized.

The method for preparing such microarray is known in the art. Such method includes the one in which a desired nucleic acid probe is deposited on a solid phase which is capable of binding to a nucleic acid (e.g. polystyrene) or which is surface-treated to be capable of binding to a nucleic acid, preferably is surface-treated to possess a cation-bearing functional group (e.g. amino, aldehyde, epoxy and the like groups), and the deposited solid phase is dried, and the one in which a desired nucleic acid probe is synthesized on the solid phase as described above. The method also includes the one in which an activated and esterified carboxyl group which has been introduced on a solid phase surface is reacted with an amino group which has been introduced at the terminal of a nucleic acid.

The nucleic acid probe to be bound to the microarray may be labeled with a labeling substance which allows the electrical or optical detection of the hybridization with a target gene in a sample. Such label is known in the art.

A molecule that can specifically bind to the protein marker can be used for the detection of the marker, making it useful in the detection of Th17 cells. Such molecule may be a nucleic acid aptamer such as DNA or RNA or an antibody that can specifically bind to the protein marker, and preferably an antibody. When the marker specific for Th17 cells is an enzyme, it can be detected by applying a substrate for the enzyme to develop color or emit light or fluorescent.

The antibody for detecting Th17 cells can be prepared by the following well-known procedure, for example. A DNA molecule encoding a protein having an amino acid sequence of the protein marker is prepared based on the nucleic acid sequence of the polynucleotide marker or the amino acid sequence of the protein marker, and is introduced into an appropriate expression vector. The obtained expression vector is introduced into an appropriate host cell, and the obtained transformed cells are cultured to obtain a desired protein. The obtained protein is purified and used as an immunogen optionally with an adjuvant to immunize an appropriate mammal such as rat or mouse. Spleen cells of the immunized animals are screened for antibody producing cells that produce an antibody directed to the target immunogen. The selected antibody producing cells are fused with myeloma cells to obtain hybridomas. These hybridomas are screened for antibody producing hybridomas that produce an antibody having specific binding property to the protein encoded by the gene. The desired antibody can be obtained by culturing the obtained antibody producing hybridomas.

The nucleic acid aptamer that can be used for detecting Th17 cells can be prepared by the following well-known procedure, for example. A nucleic acid library including random nucleic acid sequences is prepared according to a known technique, and an aptamer that specifically binds to the target protein (the protein marker) can be selected by the systematic evolution of ligands by exponential enrichment method (SELEX method) or the like.

The molecule which can specifically bind to the protein marker for detecting Th17 cells may be labeled with a labeling substance normally used in the art. The labeled antibody for detecting Th17 cells allows an easy detection of the protein marker for detecting Th17 cells, namely of Th17 cells.

The labeling substance may be the one generally used in the art including radioisotopes such as ³²P, fluorescent substances such as fluorescein, enzymes such as alkaline phosphatase and horseradish peroxidase, and biotin.

The present invention also provides a method for detecting Th17 cells by detecting the presence of at least one polynucleotide or protein marker for detecting Th17 cells as described above in a sample containing cells.

In the present method, the sample containing cells includes a biological sample obtained from mammals including human or a sample containing cultured cells. The biological sample includes blood, tissue, synovial fluid, cerebrospinal fluid, pleural fluid, ascitic fluid and the like.

An embodiment of the method for detecting the presence of the polynucleotide marker is described.

Nucleic acid (DNA or RNA) is extracted from a sample containing cells by a well-known method in the art such as the one using phenolic extraction and ethanol precipitation or a commercial DNA extraction kit.

Then, the presence of the polynucleotide marker in the obtained nucleic acid sample is detected, preferably using the nucleic acid probe for detecting Th17 cells as described above.

The polynucleotide marker can be detected by well-known methods in the art including nucleic acid amplification methods such as PCR, RT-PCT, real-time PCR, loop-mediated isothermal amplification (LAMP), hybridization methods such as Southern hybridization, Northern hybridization, fluorescence in situ hybridization (FISH), and microarray. Such methods may be carried out under the stringent condition, and the hybridization of the nucleic acid probe for detecting Th17 cells may be detected by detecting the labeling substance and the like to detect the presence of the polynucleotide marker.

The polynucleotide marker in a nucleic acid sample can also be detected by using the above microarray.

An embodiment of the method for detecting the presence of the protein marker for detecting Th17 cells is described. When the target protein marker is an intracellular protein, it is extracted from cells by using well-known methods in the art. The extraction of the protein from cells can be accomplished by well-known methods such as disruption of the cells by ultrasonication, lysis of the cells with a cell lysis solution. The protein marker in the obtained protein sample can be detected by using the molecule which specifically binds to the protein marker. Specifically, the protein marker can be detected by well-known methods in the art such as enzyme linked immunosorbent assay (ELISA) or Western blotting. The molecule which specifically binds to the protein marker in the detection is preferably the above antibody for detecting Th17 cells.

When the target protein marker is a secretory protein, the protein marker secreted in the sample containing the cells can be detected by using the molecule which specifically binds to the protein marker. Alternatively, the cells (lymphocytes) are collected from the sample and the collected cells are stimulated with anti-CD3 antibody, anti-CD28 antibody, concanavalin A, phytohemagglutinin (PHA), phorbol myristate acetate (PMA), ionomycin or the like. Then, the secreted protein marker can be detected by using the molecule which specifically binds to the protein marker. Specifically, the protein marker can be detected by well-known methods in the art such as ELISA or Western blotting. The molecule which specifically binds to the protein marker in the detection is preferably the above antibody for detecting Th17 cells.

When the target protein marker is a protein located on the cell surface, the protein marker located on the cell surface in the sample containing the cells can be detected by using the molecule which specifically binds to the protein marker. Alternatively, a membrane fraction of the cells may be obtained from the sample and the protein marker in the membrane fraction may be detected by using the molecule which specifically binds to the protein marker. Specifically, the protein marker can be detected by well-known methods in the art such as ELISA or Western blotting. When the target protein marker is a protein located on the cell surface, it can be detected by a method based on flow cytometry (FCM). The molecule which specifically binds to the protein marker in the detection is preferably the above antibody for detecting Th17 cells.

For example, the protein marker can be detected by FCM as follows.

First, the sample containing the cells is brought into contact with the antibody for detecting Th17 cells labeled with an appropriate labeling substance. Th17 cells, when exist, bind to the labeled antibody on their surfaces. Then, the sample containing the cells bound to the labeling substance can be applied to a flow cytometer to detect Th17 cells. Th17 cells that have bound to the labeling substance can optionally be classified and/or fractionated by using a cell sorter.

Such method of FCM is well-known to a person skilled in the art and he can appropriately select the reaction conditions.

### EXAMPLES

The present invention is now described in further details by way of Examples. However, it is not intended that these Examples are to limit the scope of the present invention.

### Example 1

In this Example, the genes were first selected by microarray expression analysis, which were specifically expressed in cultured Th17 cells. Then, the genes were identified among thus selected genes, which were specifically expressed in three types of disease model mice (arthritis, inflammatory bowel disease and encephalomyelitis), by expression analysis by means of real-time PCR.

(1) Expression Analysis in Cultured Th17 Cells

1-1. Isolation of Naïve T-Cells from Murine Spleen

The spleen was removed from BALB/c mice to obtain a sample containing splenocytes. After the sample was treated with ammonium chloride to lyse erythrocytes, cell fractions of CD8, B-cells, monocytes, macrophages, granulocytes and erythroblasts were removed with magnetic beads (Polyscience, Inc.) to obtain crude purified CD4 positive (CD4⁺) T-cells. From thus obtained CD4⁺ T-cells, the fraction of naive T-cells (CD4⁺/CD25^{neg}/CD44^{low}/CD62^{high}) was purified by sorting on a flow cytometer. In a similar manner, naïve T-cells were purified from splenocytes of C57/BL6 mice.

1-2. Differentiation Culture from Naïve T-Cells to Th1, Th2, Treg and Th17 Cells

Naïve T-cells which were derived from BALB/c mice and were obtained as described in the above 1. were seeded in the wells of a 24-well plate coated with anti-CD3 antibody at a density of 0.5 to 2.0×10⁶ cells/2 ml/well. Cells were incubated in T-cell medium (PRMI1640, 10% fetal bovine serum (FBS), 10 mM HEPES, 1 mM sodium pyruvate, 2 mM L-glutamic acid, 50 μM 2-mercaptoethanol, 100 U/ml penicillin and 100 mg/ml streptomycin) supplemented with cytokines and antibodies described in Table 1 and anti-CD28 antibody in an incubator at 37° C. and 5% CO₂. On third day after the start of the incubation, cytokines and antibodies described in Table 1 were added to the medium and the culture was continued for further 2 to 11 days. Thus, the differentiation was induced from naive T-cells derived from BALB/c mice to Th1, Th2, Treg and Th17 cells.

In a similar manner, the differentiation was induced from naive T-cells derived from C57/BL6 mice obtained as described in the above 1. to Th1, Th2, Treg and Th17 cells.

TABLE 1

| | Cytokine | Manufacturer | Antibody | Clone | Manufacturer |
|---|---|---|---|---|---|
| Th1 cells | IL-2 | Becton | Anti-IL-4 antibody | 11B11 | eBioscience |
| | | Dickinson | | | |
| | | (BD) | | | |
| | IL-12 | BD | | | |
| Th2 cells | IL-2 | BD | Anti-IFNγ antibody | R4-6A2 | eBioscience |
| | IL-4 | R&D SYSTEM | | | |
| Treg cells | IL-2 | BD | Anti-IL-6 antibody | MP5-20F3 | BD |
| | TGFβ | R&D SYSTEM | Anti-IFNγ antibody | R4-6A2 | eBioscience |
| | | | Anti-IL-4 antibody | 11B11 | eBioscience |
| Th17 cells | IL-6 | BD | Anti-IL-2 antibody | S4B6 | BD |
| | TGFβ | R&D SYSTEM | Anti-IFNγ antibody | R4-6A2 | eBioscience |
| | IL-23 | R&D SYSTEM | Anti-IL-4 antibody | 11B11 | eBioscience |
| | IL-1β | eBioscience | | | |
| | TNFα | eBioscience | | | |

### 1-3. Verification of Cell Differentiation by Flow Cytometer

To the suspensions of the cells which were differentiation cultured as described in the above 2. (2.5×10⁵ cells) were added phorbol myristate acetate (PMA; 50 ng/ml) and ionomycin (1 μM) to stimulate cells. Brefeldin A (10 μg/ml) was added after four hours and the incubation was continued for further two hours. The cells were washed with phosphate buffered saline (PBS) and fixed with 4% paraformaldehyde. After fixing, cells were treated with a saponin buffer (0.5% saponin, 0.5% bovine serum albumin (BSA) and 1 mM sodium azide in PBS) to increase the permeability of cell membranes. Cells were then reacted with anti-IFN-γ, anti-IL-4 and anti-IL-17 antibodies. After the reaction, cells were washed with the saponin buffer and then with PBS containing 0.5% BSA, analyzed on FACS Canto II (BD Biosciences) to verify the differentiation to Th1, Th2, Treg and Th17 cells, respectively.

### 1-4. Extraction of Total RNA

Th1, Th2, Treg and Th17 cells derived from BALB/c mice, respectively, which were cultured for 5 days as the above 2, were washed with PBS, centrifuged to pellets and stored at −80° C. Total RNA was extracted from the pellets with RNeasy Plus Mini Kit (QIAGEN) and stored at −80° C. until it was analyzed. In a similar manner, total RNA was extracted from Th1, Th2, Treg and Th17 cells derived from C57/BL6 mice respectively, which were cultured for 5 days as the above 2.

The extraction of total RNA was carried out according to the instructions attached to the Kit.

### 1-5. Microarray Expression Analysis

Total RNA (1 to 5 μg) which was extracted as described in the above 4. was reverse-transcribed to cDNA and further transcribed to biotinylated cRNA with One-Cycle Target Labeling and Control Reagents (Affymetrix). To GeneChip Mouse Genome 430 2.0 Array (Affymetrix) was added 15 μg of biotinylated cRNA, and hybridization was carried out in GeneChip Hybridization Oven 640 (Affymetrix) under the conditions of 45° C. and 60 rpm for 16 hours. After the completion of the hybridization, the microarray was washed and fluorescence labeled in GeneChip Fluidic Station 450 (Affymetrix), and scanned on GeneChip Scanner 3000 7G (Affymetrix) to obtain the fluorescent intensity data.

All operations above were carried out according to the instructions attached to the reagents and instruments.

1-6. Selection of Genes which are Specifically Expressed in Murine Th17 Cells

The fluorescent intensity data obtained as described in the above 5. was standardized with an expression analysis software Array Assist (MediBIC Group, K.K.). The fluorescent intensity of respective genes was divided by the fluorescent intensity of a housekeeping gene, glyceraldehyde 3-phosphate dehydrogenase (Gapdh) gene to obtain the relative fluorescent intensity. The relative fluorescence intensities of the genes in Th17 cells were compared to those in Th1, Th2 and Treg cells. The genes (128 genes) were selected which have the relative fluorescent intensity at least three times higher than all of those in Th1, Th2 and Treg cells in at least one of BALB/c and C57/BL6 mice (data not shown).

(2) Generation of Disease Model Mice and Preparation of Total RNA

### 2-1. Generation of SKG Arthritis Model Mice

Arthritis model mice were generated according to the following procedures.

### (i) Preparation of Bacterial Cell Components

Laminarin (from Laminaria digitata, SIGMA) was dissolved in PBS (phosphate buffered saline) to the concentration of 150 mg/ml. Curdlan (from Alcaligenes faecalis, SIGMA) was suspended in PBS (phosphate buffered saline) to the concentration of 50 mg/ml.

### (ii) Administration of Bacterial Cell Components

Female SKG spontaneously arthritis mice (7- to 8-week old) were intraperitoneally administered with laminarin (30 mg/200 μl/mouse) or curdlan (10 mg/200 μl/mouse), which were prepared in (i). After 4 weeks, curdlan (10 mg/200 μl/mouse) prepared in (i) was further administered intraperitoneally.

(iii) Evaluation of Severity of Arthritis

Symptoms of arthritis appear 30 days or more after the bacterial cell component administration. Severity was evaluated according to the following scores.
Score 0: Normal
Score 0.1: Swelling and rush at finger joints
Score 1: Mild swelling and rush at limb joints
Score 2: Severe swelling and rush at joints of forelimbs and hindlimbs

The individuals were used for further analysis which had 9 or more points for the sum of the scores for four limbs.

### (iv) Removal of Joint Tissue and Extraction of Total RNA

The skin at the joint was removed with scissors, toes were separated and limb joint tissues were removed. The obtained limb joint tissues were frozen and stored in liquid nitrogen. Total RNA was extracted from the frozen limb joint tissues with RNeasy Plus Mini kit (QIAGEN) and QIAshredder (QIAGEN).

The extraction of total RNA was carried out according to the instructions attached to the Kits.

### 2-2. Generation of SCID Implanted Colitis Model Mice (Enteritis Model Mice)

Colitis model mice were generated according to the following procedures.

(i) Preparation of Naïve CD4 T-Cells

The spleen was removed from 8- to 10-weeks old BALB/c mice to obtain a sample containing splenocytes. After the sample was treated with ammonium chloride to lyse erythrocytes, cell fractions of CD8, B-cells, monocytes, macrophages, granulocytes and erythroblasts were removed with magnetic beads (Polyscience, Inc.) to obtain crude purified CD4 positive (CD4⁺) T-cells (sample A). From thus obtained CD4⁺ T-cells, the fraction of naïve T-cells (CD4⁺/CD25^{neg}/CD44^{low}/CD62^{high} or CD4⁺/CD45RB^{high} cells) was purified by sorting on a flow cytometer.

(ii) Administration of Naïve CD4 T-Cells

C.B.17/Icr-Prkdc (scid)/CrlCrlj mice (SCID mice, 8- to 10-week old) were intraperitoneally administered with the fractionated naïve CD4 T-cells (5×10⁵ cells/300 μl/mouse).

(iii) Confirmation of Disease Onset

The change in the body weight of mice was monitored after the administration of naïve CD4 T-cells. After four weeks or more, the decrease in the body weight due to induction of enteritis was observed. The individuals whose body weight was decreased to 80% or less of the initial body weight were regarded as those having severe enteritis.

### (iv) Removal of Intestinal Tract Tissue and Extraction of Total RNA

The large intestine was removed from the severely affected individuals, three fragments corresponding to the ascending, transverse and descending colons were separated and frozen and stored in liquid nitrogen. Total RNA was extracted from the frozen intestinal tract tissues with RNeasy Plus Mini kit (QIAGEN) and QIAshredder (QIAGEN).

The extraction of total RNA was carried out according to the instructions attached to the Kits.

### 2-3. Generation of Experimental Allergic Encephalomyelitis (EAE) (Acute) Model Mice (Encephalitis Model Mice)

Acute EAE model mice were generated according to the following procedures.

### (i) Preparation of Antigen Emulsion

Incomplete Freund's adjuvant (Difco Laboratories) and the cell components of Mycobacterium tuberculosis H37Ra (Difco Laboratories) were mixed to obtain complete Freund's adjuvant (CFA; 20 mg/ml). PLP (139-151) peptide (Bio-synthesis, Inc.) dissolved in PBS (phosphate buffered saline) at 2 mg/ml was mixed with CFA in equal quantities by moving back and forth a syringe equipped with a double hub needle (Double Hub Needles set, Techno Chemical Corporation) to prepare an antigen emulsion.

### (ii) Administration of Antigen Emulsion

Female SJL mice (8- to 10-week old) were shaved at their back with hair clippers and subcutaneously administered with 50 μl of the antigen emulsion (total 100 μl) using a 1-ml syringe at two positions, i.e. left and right sides of the midline of the waist. On the same day, mice were intraperitoneally administered with Pertussis Toxin (List Biological Laboratories) (100 ng dissolved in 200 μl PBS). As healthy mice against encephalomyelitis model mice, SJL mice were used to which Pertussis Toxin only was administered.

(iii) Evaluation of Severity of Encephalomyelitis

Symptoms of encephalomyelitis appear 10 to 21 days after the antigen emulsion administration. Severity was evaluated according to the following scores.
Score 1: Tail paralysis
Score 2: Hindlimb paresis
Score 3: Hindlimb paralysis
Score 4: Forelimb paralysis
Score 5: Moribundity or death due to general paralysis

The individuals were used for further analysis which had score 2 or more.

### (iv) Extraction of Cerebrospinal Tissue and Total RNA

The cranial bone was removed from head and brain was taken out. The spinal column without head and tail was removed, PBS was injected from the vertebral foramen of the vertebrae coccygea and the spinal cord was removed by injection pressure. The obtained brain and spinal cord were frozen in liquid nitrogen. The frozen cerebrospinal tissues were homogenized with a homogenizer (AS ONE Corporation), and total RNA was extracted with RNeasy Plus Mini kit (QIAGEN) and QIAshredder (QIAGEN).

The extraction of total RNA was carried out according to the instructions attached to the Kits.

(3) Expression Analysis of Genes in Disease Model Mice by Real-Time PCR

Total RNA (2.0 to 5.0 μg) extracted from the tissue samples from disease model mice prepared as described in the above (2) was reverse-transcribed with poly dT primer (Hokkaido System Science Co., Ltd.), random primer (Hokkaido System Science Co., Ltd.) and SuperScript III reverse transcriptase (Invitrogen Corporation) to obtain cDNA. Primer sets for the genes selected in the above (1) were also prepared. The expression levels of the genes (threshold cycle values: Ct values) were measured with the obtained cDNAs as templates, the respective primer sets and Power SYBR Green PCR Master Mix (Applied Biosystems) in 7300 Real Time PCR System (Applied Biosystems). Data correction was carried out with Gapdh gene as an internal standard gene, in order to compare the results between the samples. The values representing the gene expression levels were calculated as follows:

(Gene expression level)=100000×2^{-x}

wherein X=(Ct value of a gene)−(Ct value of Gapdh gene).

The above operations were carried out according to the instructions attached to the reagents and instruments.

The above primer sets were designed with Primer3 software.

Similar experiments were carried out with the following healthy mice instead of the disease model mice.

Healthy mice against arthritis model mice: 2 BALB/c mice.

Healthy mice against colitis model mice: 2 SCID mice to which CD4 T-cells before separation of naive CD4 T-cells (sample A) were intraperitoneally administered; 2 SCID mice to which the medium was intraperitoneally administered; and 2 BALB/c mice (total 6 mice).

Healthy mice against encephalomyelitis model mice: 3 SJL mice to which Pertussis Toxin was intraperitoneally administered.

Further, for the purpose of comparison, similar experiments were carried out with the primer sets designed for the genes which have been conventionally known for their specific expression in Th17 cells (1123r, 1117f and 1119).

(4) Analysis

With regard to the genes whose expression levels were analyzed as described in the above (3), “values of the expression level in disease model mice (A) to (C)” and “ratios of the expression level in disease model mice relative to the expression level in healthy mice (A) to (C)” were calculated as follows.

### 4-1. Arthritis Model

Number of analyzed mice: 2 healthy mice and 3 arthritis model mice

Analyzed samples: Hindlimb joint tissues (two for each mouse from left and right hindlimbs)

### (i) Calculation of the Ratio of the Expression Level in Arthritis Model Mice Relative to the Expression Level in Healthy Mice (A)

The average of the expression levels was calculated from 4 samples obtained from healthy mice (left and right hindlimb joint tissues from 2 mice).

The ratio of the expression level in arthritis model mice relative to the average expression level in healthy mice, i.e. “(expression level in arthritis model mice)/(average expression level in healthy mice)” was calculated for each of 6 samples (left and right hindlimb joint tissues from 3 mice).

The ratios of the expression levels thus calculated for 6 samples were averaged to finally obtain the ratio of the expression level in arthritis model mice relative to the expression level in healthy mice (A).

### (ii) Calculation of the Expression Level in Arthritis Model Mice (A)

The average of the expression levels (Ct values) of 6 samples obtained from arthritis model mice (left and right hindlimb joint tissues from 3 mice) was calculated to obtain the expression level in arthritis model mice (A).

### 4-2. Colitis Model Mice

Number of analyzed mice: 6 healthy mice and 3 colitis model mice

Analyzed samples: three large intestinal parts corresponding to the ascending, transverse and descending colons

### (i) Calculation of the Ratio of the Expression Level in Colitis Model Mice Relative to the Expression Level in Healthy Mice (B)

For each of the large intestinal parts, the average expression level in 6 healthy mice and the average expression level in 3 colitis model mice were calculated.

The ratio of the average expression level in colitis model mice relative to the average expression level in healthy mice, i.e. “(average expression level in colitis model mice)/(average expression level in healthy mice)” was calculated for each of the three parts of the large intestine. The maximum ratio of the expression level among those calculated for the three parts of the large intestine was finally regarded as the ratio of the expression level in colitis model mice relative to the expression level in healthy mice (B).

### (ii) Calculation of the Expression Level in Colitis Model Mice (B)

The average expression level for three colitis model mice in the part which showed the maximum value of the ratio of the expression level in colitis model mice (B) as described in the above (i) was finally regarded as the expression level in colitis model mice (B).

### 4-3. Encephalomyelitis Model

Number of analyzed mice: 3 healthy mice and 3 encephalomyelitis model mice

Analyzed samples: brain, head-side spinal cord and tail-side spinal cord (total three parts)

### (i) Calculation of the Ratio of the Expression Level in Encephalomyelitis Model Mice Relative to the Expression Level in Healthy Mice (C)

For each of the above three parts, the average expression level in 3 healthy mice and the average expression level in 3 encephalomyelitis model mice were calculated.

The ratio of the average expression level in encephalomyelitis model mice relative to the average expression level in healthy mice, i.e. “(average expression level in encephalomyelitis model mice)/(average expression level in healthy mice)” was calculated for each of the three parts.

The maximum ratio of the expression level among those calculated for the three parts was finally regarded as the ratio of the expression level in encephalomyelitis model mice relative to the expression level in healthy mice (C).

### (ii) Calculation of the Expression Level in Encephalomyelitis Model Mice (C)

The average expression level for three encephalomyelitis model mice in the part which showed the maximum value of the ratio of the expression level in encephalomyelitis model mice (C) as described in the above (i) was finally regarded as the expression level in encephalomyelitis model mice (C).

(5) Results

Among 128 genes which were selected for their specific expression in the cultured Th17 cells, the genes are shown in Table 2 which have two times or more higher ratio of the expression level in disease model mice relative to the expression level in healthy mice, i.e. which show the value 2 or more for all of the ratios of the expression level (A) to (C) as obtained in the above (4).

These 27 genes were identified as those specifically expressed in Th17 cells-related disease model mice. For these identified 27 genes, the expression levels (A) to (C) and the ratios of the expression levels (A) to (C) calculated as described in the above (4) are shown in Table 2. The results of the genes which have been conventionally known for their specific expression in Th17 cells (Il23r, Il17f and Il19) are also shown in Table 2 as comparisons. The primer sets used as described in the above (3) for those identified 27 genes are shown in Table 3.

Among 27 genes shown in Table 2, Ccl20, Il17a, Il22 and RORγt (shown in italics in Table 2) are the genes which have been known for their specific expression in Th17 cells. Thus, 23 genes excluding these four genes are now identified as the genes which are specifically expressed in Th17 cells and also specifically expressed in Th17 cell-related disease model mice.

These 23 genes now identified would be useful novel markers for the detection of Th17 cells. Among these genes, the ones having higher expression levels (preferably 1,000 or more, and more preferably 10,000 or more of the expression level) are considered to be promising as the markers.

TABLE 2

| | Arthritis model | | Enteritis model | | Encephalitis model | |
|---|---|---|---|---|---|---|
| | Expression | Ratio of | Expression | Ratio of | Expression | Ratio of |
| | level (A) | expression | level (B) | expression | level (C) | expression |
| Gene symbol | (Ct value) | level (A) | (Ct value) | level (B) | (Ct value) | level (C) |
| Fcgr2b | 23646.4 | 18.4 | 3359.6 | 14.3 | 7672.1 | 78.1 |
| Klrb1f | 549.7 | 22.2 | 64.9 | 5.6 | 6.1 | 34.9 |
| Cnr2 | 2672.8 | 12.6 | 55.1 | 6.0 | 343.1 | 28.6 |
| Il27ra | 144.9 | 7.9 | 76.1 | 9.9 | 101.1 | 14.9 |
| Ebi2 | 3003.8 | 3.8 | 624.8 | 6.6 | 924.9 | 4.5 |
| Pdpn | 30735.4 | 15.7 | 2784.7 | 4.6 | 4166.2 | 3.0 |
| Tmem176b | 9374.7 | 9.9 | 5400.4 | 2.1 | 3068.7 | 7.6 |
| Tmem176a | 26470.3 | 10.2 | 14966.3 | 2.2 | 12063.5 | 5.1 |
| Il17a | 74.7 | 33.1 | 241.2 | 390.9 | 336.3 | 119304.3 |
| Il22 | 91.4 | 77.2 | 472.3 | 536.8 | 28.8 | 832.4 |
| Timp1 | 31596.0 | 27.0 | 885.7 | 31.9 | 5099.4 | 452.5 |
| Apol7b or | 1191.0 | 258.5 | 963.2 | 167.9 | 103.2 | 44.9 |
| Apol7e | | | | | | |
| Mmp13 | 37872.1 | 30.4 | 865.8 | 14.1 | 87.1 | 17.3 |
| Mcpt1 | 106.0 | 4.8 | 7007.6 | 61.7 | 1.7 | ∞ |
| Ccl20 | 613.3 | 5.1 | 6695.7 | 6.4 | 2.3 | 7.4 |
| Tgfbi | 25773.4 | 4.5 | 2725.5 | 2.3 | 6073.2 | 28.4 |
| Crispld2 | 6295.8 | 3.8 | 905.4 | 2.1 | 823.5 | 4.4 |
| Upp1 | 3529.8 | 58.3 | 37409.6 | 8.7 | 592.3 | 9.9 |
| Rbp1 | 11568.8 | 16.5 | 4900.5 | 48.9 | 2477.4 | 9.3 |
| RORyt | 65.1 | 5.7 | 105.1 | 7.8 | 67.1 | 1167.3 |
| Cyp1b1 | 1311.1 | 2.3 | 5.9 | 7.5 | 282.1 | 6.9 |
| B3gnt8 | 861.7 | 2.3 | 119.2 | 2.0 | 211.2 | 6.4 |
| Bcl2a1 | 73581.8 | 39.0 | 27636.3 | 42.2 | 16880.5 | 52.6 |
| AI447357 | 1023.4 | 9.9 | 59.0 | 3.2 | 135.8 | 5.2 |
| AK143436 | 1631.8 | 7.6 | 69.3 | 16.1 | 41.8 | 2.2 |
| 1300007F04 | 1227.2 | 3.5 | 98.5 | 2.5 | 368.5 | 4.3 |
| Rik | | | | | | |
| Hmga2-ps1 | 4349.2 | 4.0 | 212.5 | 3.7 | 552.4 | 2.8 |

(Comparison)

| | | | | | | |
|---|---|---|---|---|---|---|
| Il23r | 377.0 | 63.0 | 44.3 | 15.5 | 35.7 | 16.7 |
| Il17f | 115.4 | 73.4 | 54.8 | 29.1 | 3.7 | 48.5 |
| Il19 | 59.3 | 11.4 | 15.9 | 53.1 | 10.9 | 5.3 |

TABLE 3

| | | SEQ | | SEQ |
|---|---|---|---|---|
| Gene | Forward | ID | Reverse | ID |
| symbol | primer | NO: | primer | NO: |
| 1300007F04Rik | CCCTATGAACGTGGGTTCTTG | 1 | TTGCCGGTGTCCACTGTGT | 2 |
| Apo17b or Apo17e | GCTGCCCTGTTGCTCAAATC | 3 | ACCCAGAACAAAGAAAGAACACAAT | 4 |
| B3gnt8 | TCTTCACCCAGGCCAAACC | 5 | CAAAGAAGGTCTTCGGCACATA | 6 |
| Bcl2a1 | CAAGTAACACTACGGACCAGAGAGAA | 7 | AAATGCCAAGTGCTGATAACCA | 8 |
| Ccl20 | GCCGATGAAGCTTGTGACATT | 9 | CAGCGCACACAGATTTTCTTTT | 10 |
| Cnr2 | CAGGACAAGGCTCCACAAGAC | 11 | ATGGATGGGCTTTGGCTTCT | 12 |
| Crispld2 | CATCTTCAGGGAACTTGCTGTGT | 13 | AGGGCAGCCAGGGAACTC | 14 |
| Cyp1b1 | GCTAGCCAGCAGTGTGATGATATT | 15 | AGACAGTTCCTCACCGATGCA | 16 |
| Ebi2 | CGTCAAGTGAGTGTGTCGATCTC | 17 | TTCCCGCGAATTCTCTTCAG | 18 |
| Fcgr2b | TGGACAGCCGTGCTAAATCTT | 19 | GTTTGACCACAGCCTTTGGAA | 20 |
| Il17a | GGACTCTCCACCGCAATGAA | 21 | GCTTTCCCTCCGCATTGA | 22 |
| Il22 | CTTTCCTGACCAAACTCAGCAA | 23 | CTGGTCGTCACCGCTGATG | 24 |
| Il27ra | TCTGTGAAACTTCTGGCAAACG | 25 | TCACCTGCACACAAGGTCTTG | 26 |
| Klrb1f | TGATTGGACTCAGTGTCTTAGTGAGA | 27 | GCCACACTGCATTTTTCTATTGG | 28 |
| Mcpt1 | CCAGGTCTGTGTGGGAAGTTC | 29 | CAGAAGAGGTCCCCCAGAGTCT | 30 |
| Mmp13 | GACCCAGCCCTATCCCTTGA | 31 | GGTGCAGGCGCCAGAA | 32 |
| Pdpn | CAGGTGCTACTGGAGGGCTTA | 33 | CTTCGTCCCACGCTCTCTCT | 34 |
| Al447357 | CCTGGGCCTGCAGGAGAT | 35 | TGGTGAGATCCCTGAACAAATTAG | 36 |
| Rbp1 | CAACGTGGCCTTACGCAAA | 37 | TCGCCATCCTGCACGAT | 38 |
| RORyt | GGAGCCAAGTTCTCAGTCATGAG | 39 | TGTCCCCACAGATCTTGCAA | 40 |
| Hmga2-ps1 | TCATGTCTCCACTGTTGATTCGT | 41 | CACAGATACACAGCAGTGCAGTTC | 42 |
| Tgfbi | TCCTGGCCCCCCTGAAT | 43 | AGTCTTCATCTGGGCGTCGAT | 44 |
| Timp1 | TGCACAGTGTTTCCCTGTTTATCT | 45 | CGTCCACAAACAGTGAGTGTCA | 46 |
| Tmem176a | GGCTTCTCTCACCCCTGTATGT | 47 | CCTCTGTTTCCGCCTTTGTG | 48 |
| Tmem176b | GGTGCACGGCAGGAAAGAT | 49 | GATGTGTGGAGGCCACTTTGA | 50 |
| AK143436 | TATTGTTGCTCCTTCAGACACCTT | 51 | TTATAGCAGGTTCTGAGGTTCCCTAT | 52 |
| Upp1 | AAACGGGTGATTCGCAACA | 53 | CTGCACCAACTCCTGAACCA | 54 |

### Example 2

In this Example, the genes were first selected by microarray expression analysis, which were specifically expressed in cultured Th17 cells. Then, the genes were identified among thus selected genes, which were specifically expressed in three types of disease model mice (arthritis, inflammatory bowel disease and encephalomyelitis), by microarray expression analysis.

(1) Expression Analysis in Cultured Th17 Cells

The expression analysis in the cultured Th17 cells was carried out as described in (1) in Example 1 and 586 genes for which the expression in the Th17 cells were 1.5 times or more higher than that of all of Th1, Th2 and Treg cells were selected (data not shown).

(2) Expression Analysis of Genes in Disease Model Mice by Microarray

The expression analysis in disease model mice was carried out with microarray for 586 genes selected in the above (1).

More specifically, tissue samples were collected from three disease model mice (arthritis, enteritis and encephalitis) as described in (2) in Example 1, and total RNA was extracted from the collected tissue samples.

The extracted total RNA (1 to 5 μg for One-Cycle reagents and 10 to 100 μg for Two-Cycle reagents) was reverse-transcribed to cDNA and further transcribed to biotinylated cRNA with One-Cycle Target Labeling and Control Reagents (Affymetrix) or Two-Cycle Target Labeling and Control Reagents (Affymetrix) according to the instructions. To GeneChip Mouse Genome 430 2.0 Array (Affymetrix) was added 15 μg of biotinylated cRNA, and hybridization was carried out in GeneChip Hybridization Oven 640 (Affymetrix) under the conditions of 45° C. and 60 rpm for 16 hours. After the completion of the hybridization, the microarray was washed and fluorescence labeled in GeneChip Fluidic Station 450 (Affymetrix), and scanned on GeneChip Scanner 3000 7G (Affymetrix) to obtain the fluorescent intensity data.

A similar experiment was carried out with using healthy mice as described in Example 1 instead of disease model mice.

(3) Analysis

The fluorescent intensity data obtained was standardized on an expression analysis software Array Assist (MediBIC Group, K.K.). The fluorescent intensity of respective genes was divided by the fluorescent intensity of Gapdh gene to obtain the relative fluorescent intensity (relative fluorescence unit: RFU). The thus-calculated relative fluorescent intensities for arthritis model mice, colitis model mice and encephalomyelitis model mice were respectively regarded as the expression levels in arthritis model mice (D), in colitis model mice (E) and in encephalomyelitis model mice (F).

Next, the relative fluorescent intensity for each gene in healthy mice was compared to that in disease model mice to calculate the ratio of the relative fluorescent intensity in disease model mice relative to the relative fluorescent intensity in healthy mice, i.e. “(relative fluorescent intensity in disease model mice)/(relative fluorescent intensity in healthy mice)”. The thus-calculated ratio of the relative fluorescent intensity for arthritis model mice was regarded as the ratio of the expression level in arthritis mice relative to the expression level in healthy mice (D). The calculated ratio of the relative fluorescent intensity for colitis model mice was regarded as the ratio of the expression level in colitis mice relative to the expression level in healthy mice (E). The calculated ratio of the relative fluorescent intensity for encephalomyelitis model mice was regarded as the ratio of the expression level in encephalomyelitis mice relative to the expression level in healthy mice (F).

(4) Results

Among 586 genes which were selected for their specific expression in the cultured Th17 cells, the genes are shown in Table 4 which have two times or more higher ratio of the expression level in disease model mice relative to the expression level in healthy mice, i.e. which show the value 2 or more for all of the ratios of the expression level (D) to (F) as obtained in the above (3).

These 150 genes were identified as those specifically expressed in Th17 cells-related disease model mice. For these identified 150 genes, the measured expression levels (D) to (F) and the calculated ratios of the expression levels (D) to (F) are shown in Table 4. Table 4 further shows Unigene code of each gene; NCBI code corresponding to the amino acid sequence of the protein encoded by the gene; gene symbol; and Annotation Mapped Transcripts.

Among 150 genes shown in Table 4, Gpr15, Ccl20, Il17a, Il21 and Il22 (shown in italics in Table 4) are the genes which have been known by now for their specific expression in Th17 cells. Thus, 145 genes excluding these five genes are now identified as the genes which are specifically expressed in Th17 cells and also specifically expressed in Th17 cell-related disease model mice. These 145 genes now identified would be useful novel markers for the detection of Th17 cells. Among these genes, the ones having higher expression levels (preferably 1,000 or more, and more preferably 10,000 or more of the expression level) are considered to be promising as the markers.

TABLE 4

| | | | | | | | | Encephalomyelitis | |
|---|---|---|---|---|---|---|---|---|---|
| | | | | Arthritis model | | Enteritis model | | model | |
| | | | | Ratio of | | Ratio of | | Ratio of | Expres- |
| | | | | expres- | Expres- | expres- | Expres- | expres- | sion |
| | | | Annotation | sion | sion | sion | sion | sion | level |
| UniGene | | | Mapped | level | level (D) | level | level (E) | level | (F) |
| code | NCBI code | Gene symbol | Transcripts | (D) | (RFU) | (E) | (RFU) | (F) | (RFU) |
| Mm.34871 | NP_034626.1 | Id2 | NM_010496 | 4.4 | 1341.3 | 4.2 | 520.0 | 3.5 | 460.7 |
| Mm.5820 | NP_034957.1 | Msc | NM_010827 | 9.5 | 3654.1 | 12.2 | 2303.9 | 2.0 | 850.3 |
| Mm.390057 | NP_061293.2 | Nfat5 | NM_133957 | 9.6 | 250.1 | 3.0 | 43.3 | 10.1 | 132.0 |
| Mm.247272 | NP_085115.1 | Nfkbiz | NM_030612 | 7.4 | 37541.2 | 3.2 | 24444.9 | 10.8 | 8296.6 |
| Mm.29475 | NP_075809.1 | Plekho1 | NM_023320 | 4.0 | 9320.7 | 3.5 | 2166.0 | 3.9 | 4318.2 |
| Mm.391013 | NP_033950.1 | Runx2 | D14636 | 7.1 | 5871.1 | 10.0 | 1690.3 | 8.6 | 1128.7 |
| Mm.171615 | NP_035674.2 | Tcf12 | AK053559 | 3.5 | 2968.2 | 3.7 | 1077.2 | 2.4 | 396.4 |
| Mm.307165 | NP_036042.1 | Vax2 | NM_011912 | 3.8 | 149.2 | 3.7 | 253.1 | 4.3 | 215.2 |
| Mm.402 | NP_694799.1 | Zc3h12a | NM_153159 | 24.4 | 11110.1 | 5.2 | 7468.5 | 3.7 | 591.7 |
| — | — | Tnik | AK133568 | 4.0 | 1146.3 | 2.4 | 413.6 | 2.2 | 250.0 |
| Mm.192369 | NP_666296.1 | B3gnt8 | NM_146184 | 3.2 | 2415.2 | 3.4 | 414.6 | 5.2 | 497.1 |
| Mm.314757 | NP_076376.3 | Gcnt2 | NM_133219 | 4.6 | 1841.0 | 4.2 | 700.6 | 3.0 | 388.8 |
| Mm.272359 | NP_084097.1 | 1190003J15Rik | NM_029821 | 3.0 | 70287.0 | 3.1 | 22633.2 | 2.7 | 820.6 |
| Mm.200362 | NP_031833.3 | Cybb | NM_007807 | 86.2 | 76887.1 | 19.5 | 15150.0 | 81.4 | 19617.4 |
| Mm.31486 | NP_080681.1 | Rab32 | NM_026405 | 10.9 | 31779.2 | 2.9 | 16371.3 | 12.6 | 8094.1 |
| Mm.378888 | NP_031562.1 | Bcl2a1 | NM_007534 | 29.3 | 37849.6 | 36.0 | 59403.9 | 36.8 | 38529.4 |
| Mm.151960 | NP_084126.1 | Bcl7a | AK048827 | 4.7 | 245.7 | 5.1 | 64.1 | 2.6 | 54.3 |
| Mm.8396 | NP_031743.3 | Cmah | AK040274 | 5.6 | 1013.5 | 2.0 | 236.0 | 2.6 | 244.9 |
| Mm.240830 | NP_075607.2 | Dab2 | AK017619 | 12.3 | 3439.5 | 8.3 | 62.8 | 3.3 | 279.2 |
| Mm.5266 | NP_034314.1 | Fcer1a | NM_010184 | 10.6 | 825.7 | 45.5 | 2343.5 | 3.3 | 105.0 |
| Mm.247765 | NP_113566.2 | Nlrp4c | NM_031389 | 44.0 | 353.4 | 4.1 | 177.6 | 4.8 | 43.3 |
| Mm.279741 | XP_001473672.1 | Rbp1 | NM_011254 | 18.4 | 22582.0 | 64.9 | 34028.2 | 9.0 | 13618.3 |
| Mm.220821 | NP_619613.1 | Stab1 | NM_138672 | 13.3 | 12504.1 | 2.3 | 1317.5 | 3.9 | 797.2 |
| Mm.431927 | — | Tubb2c | NM_146116 | 9.7 | 258.2 | 4.3 | 1040.3 | 2.5 | 833.9 |
| Mm.4610 | | Upp1 | NM_009477 | 45.6 | 13544.7 | 4.5 | 122451.6 | 12.6 | 6492.1 |
| Mm.288785 | XP_130233.7 | Rc3h2 | NM_001100591 | 3.0 | 436.8 | 8.2 | 182.2 | 2.5 | 74.1 |
| — | — | Ddx6 | NM_181324 | 7.2 | 711.2 | 7.4 | 1077.0 | 9.2 | 1063.6 |
| — | — | Actr1a | AK042511 | 5.1 | 407.9 | 2.3 | 389.1 | 11.9 | 86.2 |
| Mm.374865 | NP_663461.2 | March2 | BC002144 | 304.4 | 103.9 | 3.6 | 48.6 | 3.9 | 75.3 |
| Mm.277376 | NP_038482.3 | Abca1 | NM_013454 | 8.2 | 684.9 | 3.0 | 324.4 | 2.9 | 533.7 |
| Mm.6272 | NP_034045.1 | Ccr2 | NM_009915 | 60.2 | 23738.2 | 19.4 | 6868.6 | 52.2 | 1956.4 |
| Mm.34693 | NP_061237.2 | Cd160 | NM_018767 | 19.8 | 610.8 | 21.7 | 2860.5 | 6.0 | 401.2 |
| Mm.389465 | NP_031666.2 | Cd1d2 | M63697 | 24.0 | 2596.9 | 2.6 | 519.1 | 2.7 | 66.5 |
| Mm.294439 | NP_739564.1 | Cd300a | NM_170758 | 18.0 | 31894.5 | 8.3 | 4530.5 | 5.2 | 2346.5 |
| Mm.271782 | NP_064385.1 | Clec4n | NM_020001 | 126.5 | 92759.0 | 29.3 | 41921.2 | 158.5 | 14332.7 |
| — | — | Cnr2 | NM_009924 | 93.3 | 3147.1 | 4.5 | 252.5 | 5.0 | 515.0 |
| Mm.301652 | NP_038527.2 | Cr1l | AK017702 | 10.5 | 1224.3 | 16.5 | 527.7 | 5.4 | 279.6 |
| Mm.42791 | NP_062338.2 | Crtam | NM_019465 | 10.6 | 637.4 | 8.6 | 442.8 | 6.7 | 223.7 |
| Mm.235324 | NP_031806.3 | Csf2rb | NM_007780 | 25.3 | 45439.9 | 13.2 | 11650.9 | 34.8 | 9413.5 |
| Mm.124289 | NP_109637.2 | Cxcr6 | NM_030712 | 33.3 | 3502.9 | 55.4 | 5959.5 | 411.9 | 2722.3 |
| Mm.19283 | NP_766497.3 | Cd163l1 | NM_172909 | 2.9 | 467.1 | 7.4 | 480.8 | 2.7 | 262.0 |
| Mm.425062 | NP_001070657.1 | Fcgr2b | NM_010187 | 25.4 | 50916.6 | 20.3 | 17134.9 | 46.3 | 38117.3 |
| Mm.426544 | XP_921879.1 | Gpr15 | AK017124 | 60.3 | 453.6 | 3.6 | 228.3 | 6.4 | 315.2 |
| Mm.15680 | NP_034511.2 | H2-Ea | BC106107 | 8.0 | 321937.6 | 3.7 | 130731.1 | 3.9 | 61.2 |
| Mm.38386 | NP_057880.2 | Il27ra | NM_016671 | 11.4 | 1120.6 | 6.3 | 840.5 | 4.9 | 640.5 |
| Mm.96 | NP_032425.1 | Itgae | NM_008399 | 8.3 | 3080.3 | 5.3 | 2564.7 | 3.6 | 1160.7 |
| Mm.259262 | NP_694734.1 | Klrb1f | NM_181064 | 29.5 | 180.3 | 7.0 | 23.5 | 2.1 | 15.3 |
| Mm.56899 | NP_034782.1 | Klrc1 | NM_010652 | 2.7 | 33.7 | 7.9 | 954.7 | 51.1 | 503.0 |
| Mm.347927 | NP_034782.1 | Klrc1 | AF106008 | 23.6 | 1631.9 | 15.6 | 2650.2 | 16.8 | 558.3 |
| Mm.391515 | NP_034784.1 | Klrd1 | NM_010654 | 19.6 | 4131.3 | 26.4 | 6323.5 | 37.1 | 3801.1 |
| Mm.2976 | NP_034459.2 | Pdpn | NM_010329 | 9.3 | 72451.0 | 5.0 | 11578.1 | 3.6 | 7570.9 |
| Mm.41203 | NP_766247.2 | Slc2a6 | NM_172659 | 22.9 | 6203.2 | 7.7 | 2107.6 | 2.1 | 2289.5 |
| Mm.27061 | NP_079602.4 | Tmem176a | NM_025326 | 12.2 | 18410.6 | 2.4 | 14892.4 | 3.3 | 11125.1 |
| Mm.215147 | NP_849262.1 | Tnfrsf14 | NM_178931 | 54.2 | 53.9 | 3.1 | 1147.6 | 37.1 | 955.3 |
| Mm.101198 | NP_149031.2 | Tnfrsf25 | NM_033042 | 6.3 | 2480.7 | 4.3 | 614.6 | 3.9 | 406.2 |
| Mm.25923 | NP_803416.2 | Umodl1 | NM_177465 | 3.7 | 397.9 | 2.7 | 133.7 | 2.5 | 67.2 |
| Mm.76649 | NP_035823.3 | Vcam1 | BB319726 | 7.6 | 915.0 | 9.1 | 484.9 | 4.8 | 326.2 |
| Mm.214016 | NP_034124.1 | Cyp1b1 | NM_009994 | 2.5 | 13670.3 | 2.9 | 271.1 | 5.9 | 2327.4 |
| Mm.19941 | NP_997551.1 | Acpp | NM_019807 | 3.0 | 29601.7 | 2.4 | 554.2 | 5.1 | 241.7 |
| Mm.27757 | NP_033885.2 | Bmp1 | NM_009755 | 3.0 | 14614.5 | 3.9 | 1548.9 | 2.1 | 2203.7 |
| Mm.107214 | NP_808440.1 | Bpil2 | NM_177772 | 2.6 | 59882.2 | 5.1 | 60.6 | 2.4 | 88.4 |
| Mm.116739 | NP_058656.1 | Ccl20 | NM_016960 | 15.5 | 1275.6 | 3.4 | 8381.0 | 2.2 | 37.4 |
| Mm.1282 | NP_035467.1 | Ccl3 | NM_011337 | 179.9 | 1985.9 | 108.4 | 6810.2 | 6.4 | 940.0 |
| Mm.264680 | NP_084485.1 | Crispld2 | NM_030209 | 32.0 | 87523.0 | 2.4 | 7103.1 | 2.6 | 4932.6 |
| Mm.322945 | NP_034112.2 | Ctsc | AK152524 | 12.7 | 4136.3 | 9.6 | 3231.3 | 10.8 | 2170.1 |
| Mm.14465 | NP_034501.1 | Gzmc | NM_010371 | 91.7 | 1643.5 | 135.8 | 9750.8 | 10.1 | 80.0 |
| Mm.5419 | NP_034682.1 | Il17a | NM_010552 | 21.7 | 2279.1 | 14.3 | 1592.6 | 16.2 | 1722.8 |
| Mm.14190 | XP_908431.1 | Il2 | NM_008366 | 3.3 | 190.3 | 7.9 | 420.7 | 21.8 | 259.8 |
| Mm.157689 | NP_068554.1 | Il21 | NM_021782 | 70.2 | 630.9 | 13.5 | 507.7 | 2.6 | 666.4 |
| Mm.103585 | NP_058667.1 | Il22 | NM_016971 | 13.3 | 440.3 | 75.5 | 2566.9 | 67.9 | 272.8 |
| Mm.18888 | NP_032550.2 | Lum | NM_008524 | 6.3 | 110895.0 | 3.4 | 9263.0 | 4.1 | 2107.3 |
| Mm.2639 | NP_034875.1 | Ly86 | NM_010745 | 20.8 | 17062.7 | 13.6 | 13916.8 | 9.1 | 37636.7 |
| Mm.177539 | NP_038618.1 | Lyz1 | NM_013590 | 5.7 | 166160.3 | 5.3 | 169503.4 | 30.5 | 134915.8 |
| Mm.201549 | NP_032596.1 | Mcpt1 | NM_008570 | 22.1 | 291.9 | 164.1 | 30962.1 | 7.2 | 62.0 |
| Mm.14126 | NP_062344.1 | Mmp10 | NM_019471 | 23.6 | 410.1 | 53.3 | 15899.3 | 2.2 | 204.6 |
| Mm.5022 | NP_032633.1 | Mmp13 | NM_008607 | 112.5 | 55393.1 | 28.6 | 10669.9 | 48.2 | 902.1 |
| Mm.329131 | NP_067375.3 | Prg4 | NM_021400 | 2.2 | 242058.7 | 2.5 | 286.5 | 8.2 | 822.3 |
| Mm.5033 | NP_035402 | Rln1 | AK028199 | 29.0 | 166.4 | 2.3 | 106.9 | 2.2 | 240.0 |
| Mm.21567 | NP_038678.1 | S100a8 | NM_013650 | 202.7 | 611534.7 | 1300.1 | 257905.8 | 7.6 | 47472.0 |
| Mm.14455 | NP_033395.1 | Tgfbi | NM_009369 | 4.4 | 55153.9 | 3.5 | 9576.0 | 23.5 | 13934.3 |
| Mm.8245 | NP_001037849.1 | Timp1 | NM_011593 | 41.1 | 30340.2 | 13.4 | 7035.1 | 112.7 | 34304.5 |
| Mm.440965 | NP_001020019.1 | Apol7b or | NM_001024848 | 37.8 | 236.8 | 19.2 | 301.4 | 5.9 | 280.0 |
| or | or XP_997554 | Apol7e | or | | | | | | |
| Mm.303207 | | | XM_992460 | | | | | | |
| Mm.332931 | — | 1200015M12Rik | NR_002860 | 4.2 | 32453.3 | 6.3 | 17547.3 | 3.6 | 5341.5 |
| Mm.332931 | — | 1200016E24Rik | AK075710 | 11.6 | 8042.2 | 4.9 | 460.2 | 2.1 | 1791.2 |
| Mm.432526 | — | 1300007F04Rik | ENSMUST00000100807 | 8.8 | 352.9 | 3.6 | 297.0 | 2.7 | 193.9 |
| Mm.273197 | — | 2010002N04Rik | NM_134133 | 11.4 | 7211.6 | 4.4 | 4506.5 | 3.6 | 7306.6 |
| — | — | 2900073C17Rik | XM_001473664 | 3.1 | 384.7 | 2.5 | 485.0 | 3.7 | 365.4 |
| — | — | 4930480G23Rik | AK015599 | 13.4 | 47.8 | 3.7 | 83.8 | 5.9 | 76.4 |
| Mm.148731 | — | 4933424M12Rik | AK016899 | 29.7 | 10.7 | 2.1 | 48.8 | 13.4 | 59.7 |
| — | — | 5330403D14Rik | AK017233 | 5.1 | 706.3 | 3.5 | 88.1 | 2.7 | 74.7 |
| — | — | 5430434G16Rik | AK139147 | 4.8 | 2064.4 | 3.0 | 611.2 | 2.4 | 377.6 |
| — | — | 5830444B04Rik | AK139147 | 5.4 | 32.6 | 4.1 | 323.0 | 6.7 | 83.8 |
| — | — | 9630026M06Rik | AK030886 | 6.8 | 2588.3 | 5.3 | 454.8 | 3.2 | 80.9 |
| Mm.22836 | — | A630023P12Rik | AK041600 | 31.1 | 365.1 | 7.1 | 44.6 | 15.5 | 205.4 |
| Mm.23811 | — | AI426953 | AK088689 | 2.6 | 4524.6 | 3.0 | 817.0 | 6.2 | 638.7 |
| — | — | AU015263 | AU015263 | 20.0 | 5709.8 | 14.6 | 1580.8 | 29.2 | 458.6 |
| — | — | C030044O21Rik | AK081283 | 3.7 | 62.4 | 3.8 | 116.3 | 2.6 | 150.9 |
| — | — | C230085N15Rik | AK088187 | 8.2 | 186.0 | 14.2 | 511.7 | 3.1 | 111.0 |
| Mm.158776 | NP_078771.1 | Car13 | NM_024495 | 3.5 | 2702.5 | 5.7 | 1508.2 | 2.4 | 502.7 |
| | NP_034571.1 | Hmga2-ps1 | BC064076 | 5.3 | 4711.0 | 2.2 | 304.8 | 2.2 | 735.6 |
| — | — | LOC552902 | AK047962 | 4.0 | 2311.3 | 3.7 | 1164.8 | 2.6 | 122.4 |
| Mm.279194 | NP_663602.1 | Rbm10 | AI481848 | 7.6 | 273.0 | 4.6 | 115.0 | 3.9 | 76.0 |
| Mm.29467 | NP_035402.1 | Rrad | NM_019662 | 13.2 | 3951.2 | 3.3 | 517.2 | 4.6 | 732.4 |
| Mm.262102 | NP_906271.1 | Sbno2 | NM_019662 | 17.6 | 5478.4 | 4.7 | 2080.7 | 4.3 | 835.3 |
| Mm.429434 | NP_001033008.1 | Speer1-ps1 | NR_001586 | 3.3 | 93.2 | 72.0 | 2021.3 | 55.0 | 176.9 |
| Mm.216107 | XP_133543.7 | AK086919 | AK086919 | 5.2 | 8438.1 | 3.9 | 2028.3 | 2.6 | 317.2 |
| Mm.17166 | NP_780460.2 | BG071091 | BG071091 | 2.8 | 742.0 | 4.4 | 718.9 | 3.2 | 272.9 |
| Mm.222464 | — | AK037075 | AK037075 | 3.8 | 862.5 | 10.4 | 581.0 | 4.8 | 211.6 |
| Mm.121691 | NP_780424.1 | AK048317 | AK048317 | 34.9 | 225.8 | 2.5 | 91.1 | 5.0 | 107.3 |
| Mm.330408 | NP_001103160.1 | AK081530 | AK081530 | 6.6 | 11294.4 | 9.7 | 277.2 | 3.4 | 148.2 |
| Mm.30466 | NP_114389.1 | AK143436 | AK143436 | 4.4 | 7451.1 | 25.8 | 814.7 | 3.8 | 544.1 |
| Mm.222224 | — | AW538350 | AW538350 | 5.1 | 779.1 | 5.1 | 188.7 | 9.4 | 121.5 |
| Mm.2095 | NP_033590.2 | AK138895 | AK138895 | 7.5 | 466.5 | 3.6 | 256.2 | 3.2 | 96.5 |
| Mm.309395 | NP_034292.2 | AI593283 | AI593283 | 6.0 | 2106.1 | 8.3 | 1364.1 | 3.7 | 243.0 |
| Mm.296453 | — | BM219171 | BM219171 | 19.7 | 387.4 | 5.1 | 299.4 | 2.4 | 368.2 |
| Mm.387042 | XP_001471702.1 | BB204715 | BB204715 | 46.5 | 3729.6 | 4.2 | 414.3 | 2.5 | 305.9 |
| Mm.133075 | NP_694744.1 | AI447357 | AI447357 | 10.1 | 1522.3 | 3.3 | 427.7 | 3.1 | 407.0 |
| Mm.235194 | NP_035915.2 | AK047185 | AK047185 | 7.0 | 1633.1 | 5.6 | 512.1 | 2.6 | 205.9 |
| Mm.79837 | NP_766183.1 | AW491352 | AW491352 | 3.1 | 6163.3 | 7.4 | 638.5 | 4.5 | 213.3 |
| Mm.380679 | NP_203538.2 | AK037546 | AK037546 | 33.2 | 575.1 | 2.7 | 315.6 | 9.2 | 120.2 |
| Mm.336625 | NP_604443.2 | BG072223 | BG072223 | 2.8 | 2103.5 | 2.4 | 494.6 | 2.6 | 395.0 |
| Mm.264215 | NP_997570.1 | BB283635 | BB283635 | 7.9 | 356.3 | 6.3 | 187.3 | 3.0 | 132.1 |
| Mm.256342 | NP_032475.2 | BB800733 | BB800733 | 7.2 | 612.5 | 3.8 | 26.8 | 2.2 | 201.8 |
| Mm.120650 | NP_082438.3 | AK136422 | AK136422 | 5.5 | 3494.0 | 3.4 | 251.5 | 4.5 | 197.6 |
| Mm.28615 | NP_620703.1 | BB309694 | BB309694 | 3.4 | 145.2 | 7.2 | 116.3 | 5.1 | 154.9 |
| Mm.265618 | NP_898852.2 | AI661001 | AI661001 | 37.4 | 271.5 | 4.9 | 89.3 | 5.0 | 86.2 |
| Mm.33957 | XP_992141.2 | AK080134 | AK080134 | 15.3 | 1426.1 | 5.7 | 1128.7 | 8.1 | 1113.8 |
| Mm.210815 | NP_848139.1 | AA982044 | AA982044 | 2.7 | 8681.3 | 8.0 | 798.3 | 2.2 | 343.0 |
| Mm.254873 | XP_001473339.1 | AI447438 | AI447438 | 5.7 | 5617.0 | 3.9 | 453.6 | 2.8 | 149.7 |
| Mm.387042 | XP_001471702.1 | BB204715 | BB204715 | 46.5 | 3729.6 | 4.2 | 414.3 | 2.5 | 305.9 |
| Mm.246108 | — | AK041551 | AK041551 | 22.0 | 3342.0 | 4.4 | 960.4 | 4.6 | 540.7 |
| Mm.289463 | — | AI448729 | AI448729 | 19.2 | 64.9 | 4.2 | 105.9 | 2.2 | 39.7 |
| Mm.150675 | — | BE951087 | BE951087 | 3.2 | 10.6 | 2.0 | 23.3 | 3.3 | 34.6 |
| Mm.341776 | — | AK157595 | AK157595 | 7.5 | 4366.3 | 5.8 | 1911.7 | 3.0 | 490.2 |
| Mm.255974 | — | AK085158 | AK085158 | 4.3 | 3429.3 | 4.3 | 845.7 | 2.3 | 301.8 |
| Mm.425778 | — | AK028862 | AK028862 | 2.2 | 301.8 | 2.7 | 157.7 | 3.8 | 89.5 |
| Mm.134911 | — | BG076280 | BG076280 | 5.2 | 461.4 | 3.4 | 88.3 | 2.8 | 15.4 |
| Mm.212411 | NP_083680.1 | BM115076 | BM115076 | 5.4 | 1243.8 | 2.9 | 393.8 | 2.4 | 233.9 |
| — | — | AK037590 | AK037590 | 4.1 | 825.6 | 3.8 | 210.9 | 8.1 | 160.1 |
| — | — | GENSCAN00000037577 | GENSCAN00000037577 | 11.1 | 3823.0 | 4.6 | 1141.7 | 2.6 | 559.2 |
| — | — | AK037171 | AK037171 | 3.6 | 555.0 | 2.1 | 282.5 | 2.1 | 182.2 |
| — | — | AK141370 | AK141370 | 6.9 | 44.9 | 3.1 | 192.1 | 4.6 | 157.3 |
| — | — | AK031033 | AK031033 | 34.9 | 762.3 | 29.3 | 320.8 | 5.7 | 102.3 |
| — | — | AW120969 | AW120969 | 19.3 | 293.7 | 8.8 | 502.7 | 3.0 | 430.9 |
| — | — | AI605450 | AI605450 | 3.4 | 334.7 | 3.0 | 197.4 | 2.3 | 68.6 |
| — | — | AK138320 | AK138320 | 8.4 | 1501.3 | 2.4 | 54.9 | 2.4 | 543.2 |
| Mm.393208 | — | AK149443 | AK149443 | 10.6 | 7290.4 | 2.6 | 721.7 | 3.8 | 1057.7 |
| Mm.214357 | — | AK090273 | AK090273 | 154.9 | 973.4 | 7.7 | 358.9 | 4.0 | 220.5 |
| Mm.246520 | NP_001075104.1 | BB043576 | BB043576 | 58.2 | 326.7 | 10.8 | 265.3 | 2.2 | 15.8 |
| Mm.314338 | NP_031422.3 | AK036007 | AK036007 | 2.9 | 1411.9 | 2.1 | 557.8 | 2.9 | 227.7 |

These results show that the genes shown in Table 4 are specifically expressed in Th17 cells and model mice of disease in which Th17 cells may be involved, as similar to 5 genes which have been known to be specifically expressed in Th17 cells and which are shown in italics in Table 4.

Accordingly, Th17 cells may be specifically detected by detecting the genes shown in Tables 2 and 4 by known methods in the art such as PCR or by detecting the expression of the proteins encoded by these genes by known methods in the art such as ELISA, microarray and flow cytometer methods.

### Example 3

In this Example, the expression level of Il7r gene in cultured Th cells was measured by a real-time PCR analysis.

Total RNA (2.5 μg) obtained in 1-4 in Example 1 was reverse-transcribed with poly dT primer (Hokkaido System Science Co., Ltd.), random primer (Hokkaido System Science Co., Ltd.) and SuperScript III reverse transcriptase (Invitrogen Corporation) to obtain cDNA. A primer set for Il17r gene was also prepared. The Ct value for Il7r gene was measured with the obtained cDNA as a template, the primer set and Power SYBR Green PCR Master Mix (Applied Biosystems) in 7300 Real Time PCR System (Applied Biosystems).

The above operations were carried out according to the instructions attached to the reagents and instruments. The above primer set was designed with Primer3 software.

Table 5 shows the sequences of the primer sets for Il7r and

### Gapdh Genes.

TABLE 5

| | | SEQ | | SEQ |
|---|---|---|---|---|
| Gene | Forward | ID | Reverse | ID |
| symbol | primer | NO: | primer | NO: |
| Il7r | TGAAAGCAACT | 55 | GTCGTAGTTTTCT | 56 |
| | GGACGCATGT | | CTGTGGGATTGT | |
| Gapdh | GGAGCGAGAC | 57 | GCCTTCTCCA | 58 |
| | CCCACTAACA | | TGGTGGTGAA | |

In order to compare the data between samples, Ct value was similarly obtained with using Gapdh gene as an internal standard gene. The value representing the expression level of Il7r gene was calculated as follows:

(Expression level of Ii7r gene)=100000×2^{-X}

wherein X=(Ct value of Il7r gene)−(Ct value of Gapdh gene).

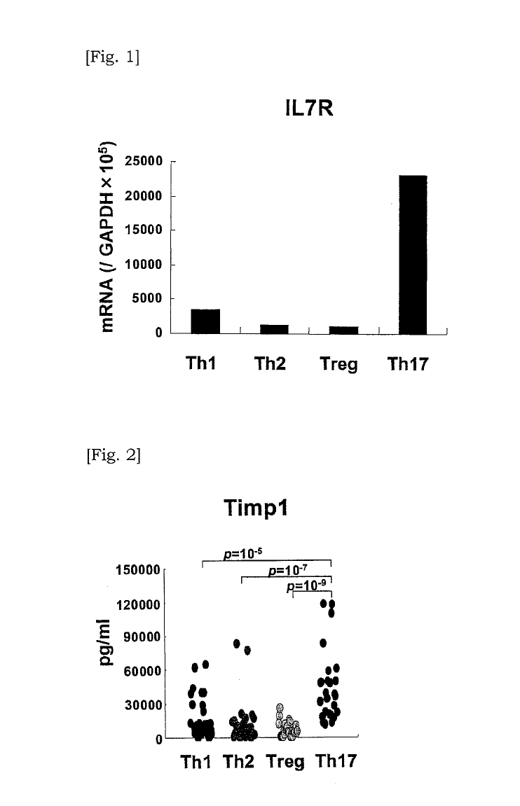

The expression levels of Il7r gene in Th1, Th2, Treg and Th17 cells derived from C57/BL6 mice are shown in FIG. 1.

FIG. 1 clearly shows that Il7r gene is strongly expressed in Th17 cells. Thus, it is demonstrated that Th17 cells could be detected with using Il7r gene as a marker.

### Example 4

In this Example, the concentration of TIMP-1 protein in culture supernatants of Th1, Th2, Treg and Th17 cells which are differentiation cultured from naïve T-cells derived from BALB/c mice was measured by ELISA.

The measurement was carried out with mouse TIMP-1 DuoSet ELISA Development kit (R&D Systems) according to the instruction attached to the kit except that casein (SIGMA-Aldrich) was added to the blocking solution to the final concentration of 0.5% and the detection was carried out with peroxidase chemiluminescence system using FEMTOGLOW Plus (Michigan Diagnostics LLC) as a luminescent substrate.

The measurements were carried out for 32 samples respectively for Th1 and Th2 cells and 28 samples respectively for Treg and Th17 cells. The significant differences (p-values) for the measured values between the cells were obtained by Mann-Whitney test. The results are shown in FIG. 2.

FIG. 2 clearly shows that TIMP-1 protein is highly secreted in Th17 cells. Thus, it is demonstrated that Th17 cells could be detected with using TIMP-1 protein as a marker.

### Example 5

In this Example, the analysis of Th17 cells using anti-TNFRSF14, anti-IL7R and anti-PDPN antibodies in flow cytometer was compared to the conventional analysis of Th17 cells using anti-CCR6 antibody in flow cytometer.

Th1, Th2, Treg and Th17 cells (10⁷ cells/ml, respectively) which were differentiation cultured in 1-2 in Example 1 were reacted with PE-Cy7-labeled anti-CD4 antibody (1.0 μg/ml, BD Biosciences), PE-labeled anti-TNFRSF14 antibody (2.0 μg/ml, eBioscience), FITC-labeled anti-IL7R antibody (1.0 μg/ml, Biolegend), anti-PDPN antibody (2.5 μg/ml, R&D Systems) and Alexa647-labeled anti-CCR6 antibody (5.0 μg/ml, Biolegend) for 20 minutes.

As anti-PDPN antibody is not fluorescent labeled, anti-goat IgG-Alexa488 antibody (1.0 μg/ml, Molecular Probes) was further reacted for 20 minutes as a fluorescent labeled secondary antibody directed to anti-PDPN antibody.

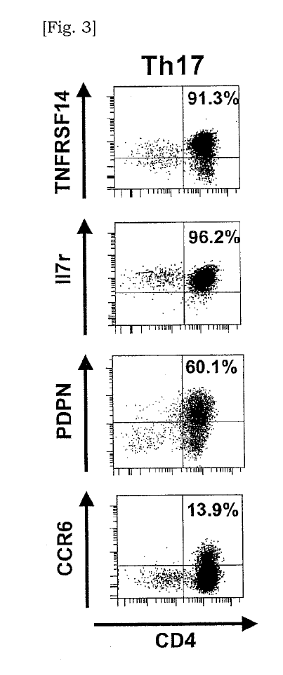

After the reaction, Th1, Th2, Treg and Th17 cells were washed with PBS containing 0.5% BSA and then suspended into PBS containing 0.5 μg/m1 7-amino-actinomycin D (7-AAD) and 0.5% BSA. The expression of the surface antigens on Th1, Th2, Treg and Th17 cells were confirmed by the analysis on FACS CantoII (BD Biosciences) with FACS DIVA software. The ratio (%) of the number of the positive cells relative to the number of total cells was calculated. FIG. 3 shows two-dimensional scattergrams of the analysis results of Th17 cells in flow cytometer with anti-CD4, anti-TNFRSF14, anti-IL7R, anti-PDPN and anti-CCR6 antibodies. Table 6 shows the ratio (%) of the number of the positive cells relative to the number of total cells for Th17 cells.

TABLE 6

| | [Number of CD4-positive- and |
|---|---|
| Analyzed | analyzed protein-positive-cells]/ |
| protein | [Number of total cells] (%) |
| TNFRSF14 | 91.3 |
| IL7R | 96.2 |
| CCR6 | 13.9 |
| PDPN | 60.1 |

FIG. 3 and Table 6 clearly show that the analyses of Th17 cells in flow cytometer with anti-TNFRSF14, anti-IL7R and anti-PDPN antibodies can detect Th17 cells in more sensitive manner than the conventional Th17 cell analysis using anti-CCR6 antibody in flow cytometer.

The present application relates to Japanese Patent Application No. 2008-218196 filed on Aug. 27, 2008, whose claims, specification, drawings and abstract are incorporated herein by reference.

1. A polynucleotide marker for detecting IL-17-producing helper T cells, which is a polynucleotide derived from at least one gene selected from:
a gene encoding an intracellular protein represented by Id2, Msc, Nfat5, Nfkbiz, Plekho1, Runx2, Tcf12, Vax2, Zc3h12a, Tnik, B3gnt8, Gcnt2, 1190003J15Rik, Cybb, Rab32, Bcl2al, Bcl7a, Cmah, Dab2, Fcer1a, Nlrp4c, Rbp1, Stab1, Tubb2c, Upp1, Rc3h2, Ddx6, Actr1a or Cyp1b1;
a gene encoding a cell membrane protein represented by March2, Abca1, Ccr2, Cd160, Cd1d2, Cd300a, Clec4n, Cnr2, Cr1l, Crtam, Csf2rb, Cxcr6, Cd163l1, Fcgr2b, H2-Ea, Il27ra, Itgae, Klrb1f, Klrc1, Klrc2, Klrd1, Pdpn, Slc2a6, Tmem176a, Tnfrsf14, Tnfrsf25, Umod, 11, Vcam1, Ebi2, Tmem176b or Il7r;
a gene encoding a secretory protein represented by Acpp, Bmp1, Bpil2, Ccl3, Crispld2, Ctsc, Gzmc, Il2, Lum, Ly86, Lyz1, Mcpt1, Mmp10, Mmp13, Prg4, Rln1, S100a8, Tgfbi, Timp1 or Apol7b or Apol7e; and
a gene represented by 1200015M12Rik, 1200016E24Rik, 1300007F04Rik, 2010002N04Rik, 2900073C17Rik, 4930480G23Rik, 4933424M12Rik, 5330403D14Rik, 5430434G16Rik, 5830444B04Rik, 9630026M06Rik, A630023P12Rik, AI426953, AU015263, C030044O21Rik, C230085N15Rik, Car13, Hmga2-ps1, LO0552902, Rbm10, Rrad, Sbno2, Speer1-ps1, AK086919, BG071091, AK037075, AK048317, AK081530, AK143436, AW538350, AK138895, AI593283, BM219171, BB204715, AI447357, AK047185, AW491352, AK037546, BG072223, BB283635, BB800733, AK136422, BB309694, AI661001, AK080134, AA982044, AI447438, BB204715, AK041551, AI448729, BE951087, AK157595, AK085158, AK028862, BG076280, BM115076, AK037590, GENSCAN00000037577, AK037171, AK141370, AK031033, AW120969, AI605450, AK138320, AK149443, AK090273, BB043576 or AK036007.

2. A protein marker for detecting IL-17-producing helper T cells consisting of at least one protein encoded by the gene according to claim 1.

3. A method for detecting IL-17-producing helper T cells characterized in that it comprises detecting the presence of at least one polynucleotide marker for detecting Th17 cells according to claim 1 in a sample containing cells.

4. A method for detecting IL-17-producing helper T cells characterized in that it comprises detecting the presence of at least one protein marker for detecting Th17 cells according to claim 2 in a sample containing cells.


SEQUENCE LISTING (58 sequences)

SEQ ID NO: 1 - DNA, 21 bp, Artificial
  PCR primer
ccctatgaacgtgggttcttg

SEQ ID NO: 2 - DNA, 19 bp, Artificial
  PCR primer
ttgccggtgtccactgtgt

SEQ ID NO: 3 - DNA, 20 bp, Artificial
  PCR primer
gctgccctgttgctcaaatc

SEQ ID NO: 4 - DNA, 25 bp, Artificial
  PCR primer
acccagaacaaagaaagaacacaat

SEQ ID NO: 5 - DNA, 19 bp, Artificial
  PCR primer
tcttcacccaggccaaacc

SEQ ID NO: 6 - DNA, 22 bp, Artificial
  PCR primer
caaagaaggtcttcggcacata

SEQ ID NO: 7 - DNA, 26 bp, Artificial
  PCR primer
caagtaacactacggaccagagagaa

SEQ ID NO: 8 - DNA, 22 bp, Artificial
  PCR primer
aaatgccaagtgctgataacca

SEQ ID NO: 9 - DNA, 21 bp, Artificial
  PCR primer
gccgatgaagcttgtgacatt

SEQ ID NO: 10 - DNA, 22 bp, Artificial
  PCR primer
cagcgcacacagattttctttt

SEQ ID NO: 11 - DNA, 21 bp, Artificial
  PCR primer
caggacaaggctccacaagac

SEQ ID NO: 12 - DNA, 20 bp, Artificial
  PCR primer
atggatgggctttggcttct

SEQ ID NO: 13 - DNA, 23 bp, Artificial
  PCR primer
catcttcagggaacttgctgtgt

SEQ ID NO: 14 - DNA, 18 bp, Artificial
  PCR primer
agggcagccagggaactc

SEQ ID NO: 15 - DNA, 24 bp, Artificial
  PCR primer
gctagccagcagtgtgatgatatt

SEQ ID NO: 16 - DNA, 21 bp, Artificial
  PCR primer
agacagttcctcaccgatgca

SEQ ID NO: 17 - DNA, 23 bp, Artificial
  PCR primer
cgtcaagtgagtgtgtcgatctc

SEQ ID NO: 18 - DNA, 20 bp, Artificial
  PCR primer
ttcccgcgaattctcttcag

SEQ ID NO: 19 - DNA, 21 bp, Artificial
  PCR primer
tggacagccgtgctaaatctt

SEQ ID NO: 20 - DNA, 21 bp, Artificial
  PCR primer
gtttgaccacagcctttggaa

SEQ ID NO: 21 - DNA, 20 bp, Artificial
  PCR primer
ggactctccaccgcaatgaa

SEQ ID NO: 22 - DNA, 18 bp, Artificial
  PCR primer
gctttccctccgcattga

SEQ ID NO: 23 - DNA, 22 bp, Artificial
  PCR primer
ctttcctgaccaaactcagcaa

SEQ ID NO: 24 - DNA, 19 bp, Artificial
  PCR primer
ctggtcgtcaccgctgatg

SEQ ID NO: 25 - DNA, 22 bp, Artificial
  PCR primer
tctgtgaaacttctggcaaacg

SEQ ID NO: 26 - DNA, 21 bp, Artificial
  PCR primer
tcacctgcacacaaggtcttg

SEQ ID NO: 27 - DNA, 26 bp, Artificial
  PCR primer
tgattggactcagtgtcttagtgaga

SEQ ID NO: 28 - DNA, 23 bp, Artificial
  PCR primer
gccacactgcatttttctattgg

SEQ ID NO: 29 - DNA, 21 bp, Artificial
  PCR primer
ccaggtctgtgtgggaagttc

SEQ ID NO: 30 - DNA, 22 bp, Artificial
  PCR primer
cagaagaggtcccccagagtct

SEQ ID NO: 31 - DNA, 20 bp, Artificial
  PCR primer
gacccagccctatcccttga

SEQ ID NO: 32 - DNA, 16 bp, Artificial
  PCR primer
ggtgcaggcgccagaa

SEQ ID NO: 33 - DNA, 21 bp, Artificial
  PCR primer
caggtgctactggagggctta

SEQ ID NO: 34 - DNA, 20 bp, Artificial
  PCR primer
cttcgtcccacgctctctct

SEQ ID NO: 35 - DNA, 18 bp, Artificial
  PCR primer
cctgggcctgcaggagat

SEQ ID NO: 36 - DNA, 24 bp, Artificial
  PCR primer
tggtgagatccctgaacaaattag

SEQ ID NO: 37 - DNA, 19 bp, Artificial
  PCR primer
caacgtggccttacgcaaa

SEQ ID NO: 38 - DNA, 17 bp, Artificial
  PCR primer
tcgccatcctgcacgat

SEQ ID NO: 39 - DNA, 23 bp, Artificial
  PCR primer
ggagccaagttctcagtcatgag

SEQ ID NO: 40 - DNA, 20 bp, Artificial
  PCR primer
tgtccccacagatcttgcaa

SEQ ID NO: 41 - DNA, 23 bp, Artificial
  PCR primer
tcatgtctccactgttgattcgt

SEQ ID NO: 42 - DNA, 24 bp, Artificial
  PCR primer
cacagatacacagcagtgcagttc

SEQ ID NO: 43 - DNA, 17 bp, Artificial
  PCR primer
tcctggcccccctgaat

SEQ ID NO: 44 - DNA, 21 bp, Artificial
  PCR primer
agtcttcatctgggcgtcgat

SEQ ID NO: 45 - DNA, 24 bp, Artificial
  PCR primer
tgcacagtgtttccctgtttatct

SEQ ID NO: 46 - DNA, 22 bp, Artificial
  PCR primer
cgtccacaaacagtgagtgtca

SEQ ID NO: 47 - DNA, 22 bp, Artificial
  PCR primer
ggcttctctcacccctgtatgt

SEQ ID NO: 48 - DNA, 20 bp, Artificial
  PCR primer
cctctgtttccgcctttgtg

SEQ ID NO: 49 - DNA, 19 bp, Artificial
  PCR primer
ggtgcacggcaggaaagat

SEQ ID NO: 50 - DNA, 21 bp, Artificial
  PCR primer
gatgtgtggaggccactttga

SEQ ID NO: 51 - DNA, 24 bp, Artificial
  PCR primer
tattgttgctccttcagacacctt

SEQ ID NO: 52 - DNA, 26 bp, Artificial
  PCR primer
ttatagcaggttctgaggttccctat

SEQ ID NO: 53 - DNA, 19 bp, Artificial
  PCR primer
aaacgggtgattcgcaaca

SEQ ID NO: 54 - DNA, 20 bp, Artificial
  PCR primer
ctgcaccaactcctgaacca

SEQ ID NO: 55 - DNA, 21 bp, Artificial
  PCR primer
tgaaagcaactggacgcatgt

SEQ ID NO: 56 - DNA, 25 bp, Artificial
  PCR primer
gtcgtagttttctctgtgggattgt

SEQ ID NO: 57 - DNA, 20 bp, Artificial
  PCR primer
ggagcgagaccccactaaca

SEQ ID NO: 58 - DNA, 20 bp, Artificial
  PCR primer
gccttctccatggtggtgaa